(19) United States
(12) Patent Application Publication
(10) Pub. No.: US 2006/0034807 A1
(43) Pub. Date: Feb. 16, 2006

(54) Innervated artificial tissues and uses thereof

(75) Inventor: May Griffith, Ontario Koa 1Lo (CA)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Ottawa Health Research Institute

(21) Appl. No.: 10/524,231

(22) PCT Filed: Aug. 11, 2003
(86) PCT No.: PCT/CA03/01191

§ 371 Date: Jul. 29, 2005

(30) Foreign Application Priority Data
Aug. 9, 2002 (CA) 2397376

(51) Int. Cl. A61K 35/30; C12N 5/08; A61K 9/14

(52) U.S. Cl. 424/93.7; 435/368; 424/486

(57) ABSTRACT

A three dimensional, innervated artificial tissue comprising a bio-synthetic matrix is provided. The artificial tissue supports cell growth, including surface coverage and three-dimensional cell in-growth, as well as nerve in-growth. Methods of preparing the innervated artificial tissue and methods of innervating artificial tissues or tissue substitutes are also provided. The artificial tissue is useful for in vitro testing of various pharmaceutical, cosmetic and household products.

### FIELD OF THE INVENTION

The present invention pertains to the field of tissue engineering and in particular to an innervated artificial tissue for in vitro testing applications.

### BACKGROUND

Currently, potential toxicological effects and eye and skin irritation potential of many chemicals, household cleaning products, cosmetics, paints and other materials are evaluated through animal testing. For example, a common way of measuring the irritancy and effect of material on the eye or skin is through the Draize test in which a material is applied directly to a rabbit's eye or skin and the irritation measured (Draize, J. H., Woodward, G. and Calvery, H. O. (1944), J. Pharm. Exp. Therapeutics, 82:377-390). A low volume test for eye irritation has been devised but this still requires living subjects. Public concern over the use of live animals in both testing and research, amongst other reasons has led to a search for alternative test methods.

The three-dimensional culture of cells to create in vitro tissue-based systems that closely mimic the natural extracellular matrix is desirable in the development of suitable alternatives to animals for toxicological and irritancy testing. A number of three dimensional cell culture systems for this purpose have been proposed. For example, International Patent Application No. PCT/CA99/00057 describes an artificial cornea constructed by layered growth of different cell lines.

In general, however, the three dimensional growth of cells is based on systems that employ hydrogels as a scaffold. U.S. Pat. No. 6,103,528, for example, describes the use of a thermally reversible gelling co-polymer for in vitro cell culture in three dimensional matrices. Cells are suspended in an aqueous solution of the hydrogel precursor, and then become entrapped within the synthetic matrix upon polymerisation.

U.S. Pat. Nos. 6,143,501, and 5,932,459 describe artificial tissues which comprise differentiated, dedifferentiated and/or undifferentiated cells in three-dimensional extracellular matrices (ECM) which are linked together. These interacting artificial tissues are described as being useful for in vitro simulation of pathogenetic and infectious processes, for establishing models of diseases, and for testing active substances.

In order to function effectively as tissue substitutes, however, three dimensional cell cultures require functional nerve in-growth. While the artificial tissues noted above are described as supporting cell growth, functional nerve cell in-growth into these artificial tissues is not described. U.S. Pat. No. 5,863,551 describes polymer matrices that can be used for treating damaged parts of the spinal cord, optic nerve or peripheral nerves. The matrices comprise a hydrogel that is a copolymer of an N-substituted methacrylamide or acrylamide, a cross-linking agent and a complex sugar or derivative, a tissue adhesion peptide or a polymer conjugate with antibodies. The polymer is described as being heterogeneous, elastically deformable and having an equilibrium water content of at least about 80%. The matrices are described for use in direct implantation into a region of damaged tissue where they are intended to interface with host tissue through a region of coarse porosity and with in-growing endogenous tissue through a region of fine porosity.

Vascularization of three dimensional cell cultures may also be important in developing an effective artificial tissue. U.S. Pat. No. 6,379,963 describes a process for vascularising a three-dimensional cell culture by inserting into the cell culture at least one vascularising tissue.

A need still exists, however, for an innervated artificial tissue suitable for in vitro use as an alternative to animal testing.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an innervated artificial tissue and uses thereof. In accordance with one aspect of the present invention, there is provided an innervated artificial tissue comprising (a) a bio-synthetic matrix comprising a synthetic polymer and a biopolymer, said synthetic polymer comprising one or more N-alkyl or N,N-dialkyl substituted acrylamide co-monomer; one or more hydrophilic co-monomer, or one or more acryl- or methacryl-carboxylic acid co-monomer derivatised to contain a pendant cross-linkable moiety, or a combination thereof; (b) a plurality of non-nerve cells associated with the bio-synthetic matrix; and (c) a plurality of functional nerve cells associated with the bio-synthetic matrix.

In accordance with another aspect of the present invention, there is provided a use of an innervated artificial tissue for in vitro toxicity, irritancy or pharmacological testing.

In accordance with another aspect of the present invention, there is provided a use of a bio-synthetic matrix for the preparation of an innervated artificial tissue, the bio-synthetic matrix comprising a synthetic polymer and a biopolymer and the synthetic polymer comprising one or more N-alkyl or N,N-dialkyl substituted acrylamide co-monomer; one or more hydrophilic co-monomer, or one or more acryl- or methacryl-carboxylic acid co-monomer derivatised to contain a pendant cross-linkable moiety, or a combination thereof

In accordance with another aspect of the present invention, there is provided a method of testing cellular effects of a substance in vitro comprising: (a) contacting an innervated artificial tissue with a test substance, said artificial tissue comprising
(i) a bio-synthetic matrix comprising a synthetic polymer and a biopolymer;
(ii) a plurality of non-nerve cells associated with the bio-synthetic matrix; and
(iii) a plurality of functional nerve cells associated with the bio-synthetic matrix, and
(b) determining the effect of the test substance on said plurality of non-nerve cells, said plurality of functional nerve cells, or both.

In accordance with another aspect of the present invention, there is provided an in vitro method of toxicology or irritancy testing of a substance comprising: (a) contacting an innervated artificial tissue with a test substance, said artificial tissue comprising
(i) a bio-synthetic matrix comprising a synthetic polymer and a biopolymer;
(ii) a plurality of non-nerve cells associated with the bio-synthetic matrix; and
(iii) a plurality of functional nerve cells associated with the bio-synthetic matrix, and
(b) determining the viability of said plurality of non-nerve cells, said plurality of functional nerve cells, or both.

In accordance with another aspect of the present invention, there is provided an in vitro method for investigation of the role of nerves in wound healing comprising: (a) creating a wound in an innervated artificial tissue, said artificial tissue comprising
(i) a bio-synthetic matrix comprising a synthetic polymer and a biopolymer;
(ii) a plurality of non-nerve cells associated with the bio-synthetic matrix; and
(ii) a plurality of functional nerve cells associated with the bio-synthetic matrix, said nerve cells being derived from said source, and
(b) comparing wound closure rates in said artificial tissue with wound closure rates in an artificial tissue that is not innervated, or in mammalian tissue.

In accordance with another aspect of the present invention, there is provided a method for the innervation of an artificial tissue comprising: (a) providing a source of nerve cells; and (b) culturing an artificial tissue in a medium in the presence of said source of nerve cells and one or more compounds that promote nerve growth, whereby nerve cells grow from said source into said artificial tissue, wherein said one or more compounds are present in said artificial tissue or in said medium, or both.

In accordance with another aspect of the present invention, there is provided a kit comprising an innervated artificial tissue of the invention and optionally instructions for use.

In accordance with another aspect of the present invention, there is provided a kit for the preparation of an innervated artificial tissue comprising: (a) a bio-synthetic matrix comprising a synthetic polymer and a biopolymer, said synthetic polymer comprising one or more N-alkyl or N,N-dialkyl substituted acrylamide co-monomer; one or more hydrophilic co-monomer, or one or more acryl- or methacryl-carboxylic acid co-monomer derivatised to contain a pendant cross-linkable moiety, or a combination thereof; and (b) optionally one or more cell lines, a source of nerve cells, instructions for use, or a combination thereof.

### BRIEF DESCRIPTION OF THE FIGURES

FIG. 1 depicts the general structure of the terpolymer of N-isopropylacrylamide, (NiPAAm), acrylic acid (AAc) and N-acryloxysuccinimide (ASI).

FIG. 2 depicts (A) transparent tissue engineered (TE) cornea with surrounding ring of opaque collagen (*). Bar, 1 cm. (B) bundles of neurites coursing through the corneal stroma to reach the targeted epithelium. Bar, 30 μm. (C) from the stroma, nerves branch to form a sub-epithelial plexus in both the fabricated cornea and human cornea (inset). Bar, 20 μm. (D) Smooth (arrowhead) and single beaded nerve fibres (arrow) that migrate into the epithelium of the TE corneas. Bar, 25 μm. (E) a nerve (arrow) invaginating an epithelial cell in the TE cornea (m=mitochondria, arrowhead=vesicles). Bar, 0.5 μm. (F) a nerve fibre penetrating an epithelial cell in the TE cornea, with dense (white arrowheads) and clear vesicles (black arrowheads). Bar, 0.4 μm.

FIG. 3 depicts nerve fibres growing into a TE cornea from the scleral scaffold, double labelled with (A) an anti-neurofilament antibody marker for nerve fibres and (B) for sodium channels. Bar, 15 μm. (C) an example of a raw, unsubtracted trace evoked by a constant voltage stimulus pulse delivered to the ganglion cell cluster and (D) a subtraction of the response obtained after lidocaine application.

FIG. 4 depicts (A) normalized total healing for TE corneas with and without DRG. (B) Epithelial cell proliferation in wounded corneas with and without innervation. (C) Substance P(SP) release over time from innervated corneas treated with 1% capsaicin versus vehicle-treated controls. (D) Normalized SP release from innervated corneas treated with 1% capsaicin or 50 μM veratridine versus controls at various time intervals post-treatment.

FIG. 5 depicts an innervated TE cornea (A) and a non-innervated control (B) treated with detergent and stained with live/dead stain. Bar, 50 μm. (C) nerves (arrowheads) and blood vessel-like structures (arrow) in a fabricated pseudo-sclera surrounding a TE cornea. Bar, 20 μm. (D) nerve growth patterns within a collagen-poly (N-isopropyl polyacrylamide) hydrogel. Bar, 20 μm.

FIG. 6 presents the results from zymographic detection of metalloproteases.

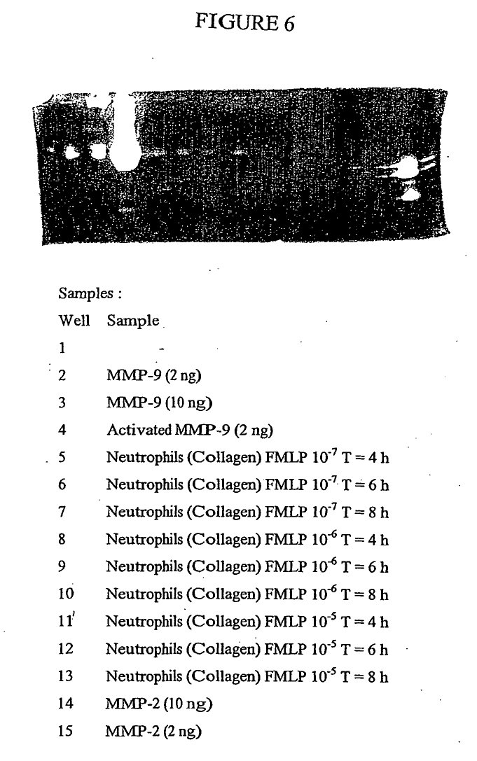

FIG. 7 presents the effects of growth factors on angiogenesis.

FIG. 8 presents the effects of retinyl acetate on angiogenesis.

FIG. 9 presents (A) the structure of a terpolymer containing a cross-linked bioactive according to one embodiment of the invention, (B) a corneal scaffold composed of cross-linked collagen and the terpolymer shown in (A), (C) shows a corneal scaffold composed of thermogelled collagen only (D) shows the number of cell layers within the stratified epithelium grown on different bio-synthetic hydrogels, (E) shows the nerve density within different hydrogels at 75 and 100 μm from the hydrogel edge.

FIG. 10 demonstrates epithelial cell growth and stratification on various hydrogels. (A) low magnification views of epithelial growth on the hydrogels. Inset is higher magnification. (B) Counts of the cell thickness of the epithelium grown over the hydrogels.

FIG. 11 depicts the results of innervation compatibility tests on various hydrogel matrices.

### DETAILED DESCRIPTION OF THE INVENTION

It should be understood that this invention is not limited to the particular process steps and materials disclosed herein, but is extended to equivalents thereof as would be recognised by those ordinarily skilled in the relevant arts. It should also be understood that terminology employed herein is for the purpose of describing particular embodiments only and is not intended to be limiting.

Definitions

Unless defined otherwise, all technical and scientific terms used herein have the same meaning as commonly understood by one of ordinary skill in the art to which this invention pertains.

The term “hydrogel” as used herein refers to a cross-linked polymeric material which exhibits the ability to swell in water or aqueous solution without dissolution and to retain a significant portion of water or aqueous solution within its structure.

The term “polymer” as used herein refers to a molecule consisting of individual monomers joined together. In the context of the present invention, a polymer may comprise monomers that are joined “end-to-end” to form a linear molecule, or may comprise monomers that are joined together to form a branched structure.

The term “monomer” as used herein refers to a simple organic molecule which is capable of forming a long chain either alone or in combination with other similar organic molecules to yield a polymer.

The term “co-polymer” as used herein refers to a polymer that comprises two or more different monomers. Co-polymers can be regular, random, block or grafted. A regular co-polymer refers to a co-polymer in which the monomers repeat in a regular pattern (e.g. for monomers A and B, a regular co-polymer may have a sequence: ABABABAB). A random co-polymer is a co-polymer in which the different monomers are arranged randomly or statistically in each individual polymer molecule (e.g. for monomers A and B, a random co-polymer may have a sequence: AABABBABBBAAB). In contrast, a block co-polymer is a co-polymer in which the different monomers are separated into discrete regions within each individual polymer molecule (e.g. for monomers A and B, a block co-polymer may have a sequence: AAABBBAAABBB). A grafted co-polymer refers to a co-polymer which is made by linking a polymer or polymers of one type to another polymer molecule of a different composition.

The term “terpolymer,” as used herein, refers to a co-polymer comprising three different monomers.

The term “bio-polymer” as used herein refers to a naturally occurring polymer. Naturally occurring polymers include, but are not limited to, proteins and carbohydrates.

The term “synthetic polymer” as used herein refers to a polymer that is not naturally occurring and that is produced by chemical or recombinant synthesis.

The terms “alkyl” and “lower alkyl” are used interchangeably herein to refer to a straight chain or branched alkyl group of one to eight carbon atoms or a cycloalkyl group of three to eight carbon atoms. These terms are further exemplified by such groups as methyl, ethyl, n-propyl, i-propyl, n-butyl, t-butyl, 1-butyl (or 2-methylpropyl), i-amyl, n-am yl, hexyl, cyclopropyl, cyclobutyl, cyclopentyl, cyclohexyl and the like.

The term “bioactive agent,” as used herein, refers to a molecule or compound which exerts a physiological, therapeutic or diagnostic effect in vivo. Bioactive agents may be organic or inorganic. Representative examples include proteins, peptides, carbohydrates, nucleic acids and fragments thereof, anti-tumour and anti-neoplastic compounds, anti-viral compounds, anti-inflammatory compounds, antibiotic compounds such as antifingal and antibacterial compounds, cholesterol lowering drugs, analgesics, contrast agents for medical diagnostic imaging, enzymes, cytokines, local anaesthetics, hormones, anti-angiogenic agents, neurotransmitters, therapeutic oligonucleotides, viral particles, vectors, growth factors, retinoids, cell adhesion factors, extracellular matrix glycoproteins (such as laminin), hormones, osteogenic factors, antibodies and antigens.

As used herein, the term “about” refers to a +/−10% variation from the nominal value. It is to be understood that such a variation is always included in any given value provided herein, whether or not it is specifically referred to.

Innervated Artificial Tissues

The present invention provides innervated artificial tissues based on bio-synthetic matrix scaffolds which support cell and nerve growth.

1. Bio-Synthetic Matrix

A bio-synthetic matrix according to the present invention comprises a synthetic polymer and a bio-polymer. The matrix is capable of supporting nerve in-growth and cell growth, including epithelial and endothelial surface coverage (i.e. two dimensional, 2D, growth) and three-dimensional (3D) cell in-growth (for example, stromal keratocyte invasion and angiogenesis). The matrix can further comprise one or more bioactive agents such as growth factors, retinoids, cell adhesion factors, laminin, and the like. The bioactive agent can be covalently attached to the synthetic polymer, or it may be encapsulated and dispersed within the final matrix. The matrix may also comprise cells encapsulated and dispersed therein, or grown on the matrix, which are capable of proliferating upon exposure to appropriate culture conditions.

In one embodiment of the invention, the bio-synthetic matrix supports growth of vascular endothelial cells. In another embodiment of the invention, the bio-synthetic matrix supports angiogenesis.

1.1 Synthetic Polymer

In accordance with the present invention, the synthetic polymer that is incorporated into the bio-synthetic matrix comprises one or more of an acrylamide derivative, a hydrophilic co-monomer and a derivatised carboxylic acid co-monomer which comprises pendant cross-linking moieties.

As used herein, an “acrylamide derivative” refers to a N-alkyl or N,N-dialkyl substituted acrylamide or methacrylamide. Examples of acrylamide derivatives suitable for use in the synthetic polymer of the present invention include, but are not limited to, N-methylacrylamide, N-ethylacrylamide, N-isopropylacrylamide (NiPAAm), N-octylacrylamide, N-cyclohexylacrylamide, N-methyl-N-ethylacrylamide, N-methylmethacrylamide, N-ethylmethacrylamide, N-isopropylmethacrylamide, N,N-dimethylacrylamide, N,N-diethylacrylamide, N,N-dimethylmethacrylamide, N,N-diethylmethacrylamide, N,N-dicyclohexylacrylamide, N-methyl-N-cyclohexylacrylamide, N-acryloylpyrrolidine, N-vinyl-2-pyrrollidinone, N-methacryloylpyrrolidine, and combinations thereof.

A “hydroplilic co-monomer” in the context of the present invention is a hydrophilic monomer that is capable of co-polymerisation with the acrylamide derivative and the derivatised carboxylic acid components of the synthetic polymer. The hydrophilic co-monomer is selected to maintain adequate solubility for polymerisation and to provide aqueous solubility of the polymer and freedom from phase transition of the final hydrogel. Examples of suitable hydrophilic co-monomers are hydrophilic acryl- or methacryl-compounds such as carboxylic acids including acrylic acid, methacrylic acid and derivatives thereof, acrylamide, methacrylamide, hydrophilic acrylamide derivatives, hydrophilic methacrylamide derivatives, hydrophilic acrylic acid esters, hydrophilic methacrylic acid esters, vinyl ethanol and its derivatives and ethylene glycols. The carboxylic acids and derivatives may be, for example, acrylic acid, methacrylic acid, 2-hydroxyethyl methacrylate (HEMA), or a combination thereof. Examples of hydrophilic acrylamide derivatives include, but are not limited to, N,N-dimethylacrylamide, N,N-diethylacrylamide, 2-[N,N-dimethylamino]ethylacrylamide, 2-[N,N-diethylamino]ethylacrylamide, N,N-diethylmethacrylamide, 2-[N,N-dimethylamino] ethylmethacrylamide, 2-[N,N-diethylamino]ethylmethacrylamide, N-vinyl-2-pyrrollidinone, or combinations thereof. Examples of hydrophilic acrylic esters include, but are not limited to, 2-[N,N-diethylamino] ethylacrylate, 2-[N,N-dimethylamino]ethylacrylate, 2-[N,N-diethylamino]ethylmethacrylate, 2-[N,N-dimethylamino]ethylmethacrylate, or combinations thereof.

As used herein, a “derivatised carboxylic acid co-monomer” refers to a hydrophilic acryl- or methacryl-carboxylic acid, for example, acrylic acid, methacrylic acid, or a substituted version thereof, which has been chemically derivatised to contain one or more cross-linking moieties, such as succinimidyl groups, imidazoles, benzotriazoles and p-nitrophenols. The term “succinimidyl group” is intended to encompass variations of the generic succinimidyl group, such as sulphosuccinimidyl groups. Other similar structures such as 2-(N-morpholino)ethanesulphonic acid will also be apparent to those skilled in the art. In the context of the present invention the group selected as a cross-linking moiety acts to increase the reactivity of the carboxylic acid group to which it is attached towards primary amines (i.e. —NH₂ groups) and thiols (i.e. —SH groups). Examples of suitable groups for derivatisation of the carboxylic acid co-monomers for use in the synthetic polymer include, but are not limited to, N-succinimide, N-succinimide-3-sulphonic acid, N-benzotriazole, N-imidazole and p-nitrophenol.

In one embodiment of the present invention, the synthetic polymer comprises one or more of:
(a) an acrylamide derivative of general formula I: 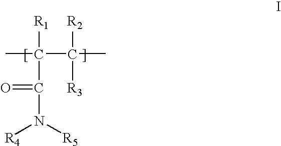
   wherein:
   R₁, R₂, R₃, R₄ and R₅ are independently selected from the group of: H and lower alkyl;
(b) a hydrophilic co-monomer having the general formula II: 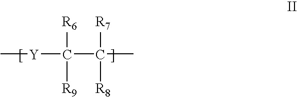
   wherein:
   Y is O or is absent;
   R₆, and R₇ are independently selected from the group of: H and lower alkyl;
   R₈ is H, lower alkyl or —OR′, where R′ is H or lower alkyl; and
   R₉ is H, lower alkyl or —C(O)R₁₀, and
   R₁₀ is —NR₄R₅ or —OR″, where R″ is H or CH₂CH₂OH;
      and (c) a derivatised carboxylic acid having the general formula III: 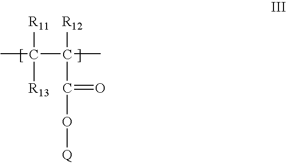
   wherein:
   R₁₁, R₁₂ and R₁₃ are independently selected from the group of: H and lower alkyl and
   Q is N-succinimido, 3-sulpho-succinimdo (sodium salt), N-benzotriazolyl, N-imidazolyl and p-nitrophenyl.

The term “lower alkyl” refers to a branched or straight chain alkyl group having 1 to 4 C atoms. This term is further exemplified by such groups as methyl, ethyl, n-propyl, i-propyl, n-butyl, t-butyl, 1-butyl (or 2-methylpropyl) and the like.

In one embodiment, the synthetic polymer comprises one or more acrylamide derivative of general formula I, one or more hydrophilic co-monomer of general formula II and one or more derivatised carboxylic acid of general formula III, as described above, wherein the term “lower alkyl” refers to a branched or straight chain alkyl group having 1 to 8 carbon atoms.

In another embodiment, the synthetic polymer comprises one or more acrylamide derivative of general formula I, one or more hydrophilic co-monomer of general formula II and one or more derivatised carboxylic acid of general formula III, as described above, wherein the term “lower alkyl” refers to to a cycloalkyl group having 3 to 8 carbon atoms, such as cyclopropyl, cyclobutyl, cyclopentyl and cyclohexyl.

The synthetic polymer should be sufficiently soluble in aqueous solution to facilitate hydrogel formation. In accordance with one embodiment of the present invention, the synthetic polymer has an aqueous solubility of at least about 0.5 weight/volume (w/v) In another embodiment, the synthetic polymer has an aqueous solubility of between about 1.0 w/v % and about 50 w/v %. In a further embodiment, the synthetic polymer has an aqueous solubility of about 5 W/v % and about 45 w/v %.

The overall hydrophilicity of the synthetic polymer is controlled to confer water solubility at a temperature between 0° C. and physiological temperatures without precipitation or phase transition. In one embodiment of the present invention, the synthetic polymer is water soluble between about 0° C. and about 37° C.

As is known in the art, most synthetic polymers have a distribution of molecular mass and various different averages of the molecular mass are often distinguished, for example, the number average molecular mass (Mₙ) and the weight average molecular mass (M_{w}). The molecular weight of a synthetic polymer is usually defined in terms of its number average molecular mass (Mₙ), which in turn is defined as the sum of nᵢMᵢ divided by the sum of nᵢ, where nᵢ is the number of molecules in the distribution with mass Mᵢ. The synthetic polymer for use in the matrix of the present invention typically has a number average molecular mass (Mₙ) between 5,000 and 1,000,000. In one embodiment of the present invention, the Mₙ of the polymer is between about 25,000 and about 80,000. In another embodiment, the Mₙ of the polymer is between about 30,000 and about 50,000. In a further embodiment, the Mn of the polymer is between about 50,000 and about 60,000.

As is known in the art, certain water-soluble polymers exhibit a lower critical solution temperature (LCST) or “cloud point.” The LCST of a polymer is the temperature at which phase separation occurs (i.e. the polymer begins to separate from the surrounding aqueous medium). Typically, for those polymers or hydrogels that are clear, the LCST also corresponds to the point at which clarity begins to be lost. It will be readily apparent that for artificial tissue applications, the presence or absence of phase separation in the final hydrogel may not be relevant provided that the hydrogel still supports cell and functional nerve growth. For other applications, however, a lack of phase separation in the final hydrogel may be critical, for example, for optical applications clarity (and, therefore, the absence of any phase transition) will be important.

Thus, in accordance with one embodiment of the present invention, synthetic polymers with a LCST between about 35° C. and about 60° C. are selected for use in the hydrogels. It is also known in the art that the LCST of a polymer may be affected by the presence of various solutes, such as ions or proteins, and by the nature of compounds cross-linked or attached to the polymer. Such effects can be determined empirically using standard techniques and selection of a synthetic polymer with an appropriate LCST for a particular application is considered to be within the ordinary skills of a worker in the art.

One skilled in the art will appreciate that the selection and ratio of the components in the synthetic polymer will be dependent to varying degrees on the final application of the bio-synthetic matrix. For example, as indicated above clarity is a major consideration for those matrices intended for ophthalmic applications, whereas for other tissue engineering applications, the clarity of the matrix may not be an important factor. Furthermore, it will be appreciated that if bioactive agents are to be covalently attached (or “grafted”) to the polymer, or if the synthetic and bio-polymers are to be cross-linked in the final hydrogel, then a synthetic polymer comprising a derivatised carboxylic acid co-monomer will be useful. In one embodiment of the invention, the final synthetic polymer comprises a plurality of pendant reactive moieties available for cross-linking, or grafting, of appropriate biomolecules.

In accordance with the present invention, the synthetic polymer can be a homopolymer, i.e. comprising repeating units of a single monomer, or it can be a co-polymer comprising two or more different monomers. Co-polymers contemplated by the present invention include linear, branched, regular, random and block co-polymers.

Homopolymers contemplated for use in the hydrogels of the present invention include homopolymers of acrylamide derivative monomers having a general formula I. An exemplary homopolymer would be a poly(NiPAAm) homopolymer. Useful co-polymers include co-polymers of different acrylamide derivatives of formula I, co-polymers of acrylamide derivatives of formula I and hydrophilic co-monomers of general formula II, co-polymers of acrylamide derivatives of formula I and derivatised carboxylic acids of general formula III and co-polymers of acrylamide derivatives of formula I, hydrophilic co-monomers of general formula II and derivatised carboxylic acids of general formula III. In order to generate a synthetic co-polymer that is suitably robust and thermostable for its intended application, the ratio of the various co-monomers in the polymer should be optimised. Accordingly, the acrylamide derivative monomers are present in the synthetic polymer in the highest molar ratio. In one embodiment of the invention, one or more the acrylamide derivative monomer(s) make up between about 75% and about 100% of the synthetic polymer, wherein the % value represents the molar %. Selection of suitable molar ratios of each component to provide a final synthetic polymer with the desired properties is within the ordinary skills of a worker in the art.

In one embodiment of the present invention, the synthetic polymer is a random or block co-polymer comprising an acrylamide derivative and a hydrophilic co-monomer. In another embodiment, the synthetic polymer is a co-polymer comprising NiPAAm monomer and acrylic acid (AAc) monomer.

In an alternative embodiment of the present invention, the synthetic polymer is a terpolymer comprising an acrylamide derivative, a hydrophilic co-monomer and a derivatised carboxylic acid co-monomer. In another embodiment, the amount of acrylamide derivative in the polymer is between 50% and 90%, the amount of hydrophilic co-monomer is between 5% and 50%, and the amount of derivatised carboxylic acid co-monomer is between 0.1% and 15%, wherein the sum of the amounts of acrylamide derivative, hydrophilic co-monomer and derivatised carboxylic acid co-monomer is 100%, wherein the % value represents the molar ratio.

In a further embodiment, the synthetic polymer is a terpolymer comprising NiPAAm monomers, acrylamide (AAm) monomers or acrylic acid (AAc) monomers and a derivatised acrylic acid monomer. In a further embodiment, the terpolymer comprises NiPAAm monomer, AAc monomer and N-acryloxysuccinimide in a ratio of about 85:10:5 molar %.

In another alternate embodiment of the invention, the synthetic polymer is a random or block co-polymer comprising an acrylamide derivative and a derivatised carboxylic acid co-monomer. In a further embodiment, the molar ratio of the acrylamide derivative is between about 50% and about 99.5% and the molar ratio of the derivatised carboxylic acid co-monomer is between about 0.5% and about 50%. In accordance with another embodiment of the invention, the molar ratio of the acrylamide derivative is between about 80% and about 99% and the molar ratio of the derivatised carboxylic acid co-monomer is between about 1% and about 20%.

In another embodiment, the synthetic polymer comprises DMAA monomer and a derivatised acrylic acid monomer. In another embodiment, a synthetic polymer comprises DMAA monomer and N-acryloxysuccinimide in a ratio of about 95:5 molar %.

1.2 Bio-Polymers

Bio-polymers are naturally-occurring polymers, such as proteins and carbohydrates. In accordance with the present invention, the bio-synthetic matrix comprises a bio-polymer or a derivatised version thereof cross-linked to the synthetic polymer by means of the pendant cross-inking moieties in the latter. Thus, for the purposes of the present invention the bio-polymer contains one or more groups which are capable of reacting with the cross-linking moiety (e.g. a primary amine or a thiol), or can be derivatised to contain such a group. Examples of suitable bio-polymers for use in the present invention include, but are not limited to, collagens (including Types I, II, III, IV and V), denatured collagens (or gelatins), fibrin-fibrinogen, elastin, glycoproteins, alginate, chitosan, hyaluronic acid, chondroitin sulphates and glycosaminoglycans (or proteoglycans). One skilled in the art will appreciate that some of these bio-polymers may need to be derivatised in order to contain a suitable reactive group as indicated above, for example, glucosaminoglycans need to be deacetylated or desulphated in order to possess a primary amine group. Such derivatisation can be achieved by standard techniques and is considered to be within the ordinary skills of a worker in the art.

Suitable bio-polymers for use in the invention can be purchased from various commercial sources or can be prepared from natural sources by standard techniques.

2. Preparation of the Bio-Synthetic Matrix

Polymerization of the components for the synthetic polymer can be achieved using standard methods known in the art [for example, see A. Ravve “Principles of Polymer Chemistry”, Chapter 3. Plenum Press, New York 1995]. Typically appropriate quantities of each of the monomers are dispersed in a suitable solvent in the presence of an initiator. The mixture is maintained at an appropriate temperature and the polymerisation reaction is allowed to proceed for a pre-determined period of time. The resulting polymer can then be purified from the mixture by conventional methods, for example, by precipitation.

The solvent for the polymerisation reaction may be a non-aqueous solvent if one or more monomer is sensitive to hydrolysis or it may be an aqueous solvent. Suitable aqueous solvents include, but are not limited to, water, buffers and salt solutions. Suitable non-aqueous solvents are typically cyclic ethers (such as dioxane), chlorinated hydrocarbons (for example, chloroform) or aromatic hydrocarbons (for example, benzene). The solvent may be nitrogen purged prior to use, if desired. In one embodiment of the present invention, the solvent is a non-aqueous solvent. In another embodiment, the solvent is dioxane.

Suitable polymerisation initiators are known in the art and are usually free-radical initiators. Examples of suitable initiators include, but are not limited to, 2,2′-azobisisobutyronitrile (AIBN), other azo compounds, such as 2,2′-azobis-2-ethylpropionitrile; 2,2′-azobis-2-cyclopropylpropionitrile; 2,2′-azobiscyclohexanenitrile; 2,2′-azobiscyclooctanenitrile, and peroxide compounds, such as dibenzoyl peroxide and its substituted analogues, and persulfates, such as sodium, potassium, and the like.

Once the synthetic polymer has been prepared, and purified if necessary, it can be characterised by various standard techniques. For example, the molar ratio composition of the polymer can be determined by nuclear magnetic resonance spectroscopy (proton and/or carbon-13) and bond structure can be determined by infrared spectroscopy. Molecular mass can be determined by gel permeation chromatography and/or high pressure liquid chromatography. Thermal characterisation of the polymer can also be conducted, if desired, for example by determination of the melting point and glass transition temperatures using differential scanning calorimetric analysis. Aqueous solution properties such as micelle and gel formation and LCST can be determined using visual observation, fluorescence spectroscopy, UV-visible spectroscopy and laser light scattering instruments.

In one embodiment of the present invention, the synthetic polymers prepared by dispersing the monomers in nitrogen-purged dioxane in the presence of the initiator AIBN and allowing polymerisation to proceed at a temperature of about 60° C. to 70° C. The resulting polymer is purified by repeated precipitation.

If cross-linking between the synthetic and bio-polymers is desired, this can also be readily achieved using standard techniques. Methods of cross-linking polymers are well-known in the art and include, for example, the use of cross-linking agents such as N-(3-dimethylaminopropyl)-N′-ethylcarbodiimide (EDC) and N-hydroxysuccinimide. Alternatively, for synthetic polymers that contain pendant cross-linking groups, cross-linking can be achieved by mixing appropriate amounts of synthetic and bio-polymer at room temperature in an appropriate solvent. Typically the solvent is an aqueous solvent, such as a salt solution, buffer solution, cell culture medium, or a diluted or modified version thereof. One skilled in the art will appreciate that in order to preserve triple helix structure of polymers such as collagen and to prevent fibrillogenisis and/or opacification of the hydrogel, the cross-linking reaction should be conducted in aqueous media with close control of the pH and temperature. The significant levels of amino acids in nutrient media normally used for cell culture can cause side reactions with the cross-linking moieties of the synthetic polymer, which can result in diversion of these groups from the cross-linking reaction. Use of a medium free of amino acids and other proteinaceous materials can help to prevent these side reactions and, therefore, increase the number of cross-links that form between the synthetic and bio-polymers. Conducting the cross-linking reaction in aqueous solution at room or physiological temperatures allows both cross-linking and the much slower hydrolysis of any residual cross-linking groups to take place.

Alternatively, a termination step can be included to react any residual cross-liking groups in the matrix. For example, one or more wash steps in a suitable buffer containing glycine will terminate any residual cross-linking groups as well as removing any side products generated during the cross-linking reaction. Unreacted cross-linking groups may also be terminated with a polyfunctional amine such as lysine or triethylenetetraamine leading to formation of additional short, inter-chain cross-links. Wash steps using buffer alone can also be conducted if desired in order to remove any side products from the cross-linking reaction. If necessary, after the cross-linking step, the temperature of the cross-linked polymer suspension can be raised to allow the hydrogel to form fully.

One skilled in the art will understand that the amount of each polymer to be included in the hydrogel will be dependent on the choice of polymers and the intended application for the hydrogel. In general, using higher initial amounts of each polymer will result in the formation of a more robust gel due to the lower water content. The presence of cross-links will also strengthen the hydrogel and alter its elasticity. Higher quantities of water or aqueous solvent will produce a soft hydrogel. In one embodiment of the present invention, the final hydrogel contains about 95% by weight of water or aqueous solvent.

In accordance with another embodiment of the present invention, the final hydrogel comprises between about 40 and 99.6% by weight of water or aqueous solvent, between about 0.1 and 30% by weight of synthetic polymer and between about 0.3 and 30% by weight of bio-polymer.

In one embodiment of the present invention, the final hydrogel comprises between about 80 and 98.5% by weight of water or aqueous solvent, between about 0.5 and 5% by weight of synthetic polymer and between about 1 and 15% by weight of bio-polymer. In another embodiment, the final hydrogel contains about 95 to 97% by weight of water or aqueous solvent and between about 1-2% by weight of synthetic polymer and about 2-3% by weight of bio-polymer. In a further embodiment, the final hydrogel contains about 94 to 98% by weight of water or aqueous solvent and between about 1-3% by weight of synthetic polymer and about 1-3% by weight of bio-polymer.

Similarly, the relative amounts of each polymer to be included in the hydrogel will be dependent on the type of synthetic polymer and bio-polymer being used and upon the intended application for the hydrogel. One skilled in the art will appreciate that the relative amounts bio-polymer and synthetic polymer will influence the final gel properties in various ways, for example, high quantities of bio-polymer will produce a very stiff hydrogel and high concentrations of synthetic polymer will produce an opaque hydrogel. In accordance with the present invention, the weight per weight (w/w) ratio of synthetic polymer: bio-polymer is between about 1:0.07 and about 1:14.

In one embodiment of the present invention, the w/w ratio of synthetic polymer: bio-polymer is between 1:1.3 and 1:7. In another embodiment, the w/w ratio of synthetic polymer: bio-polymer is between 1:1 and 1:3. In a further embodiment, the w/w ratio of synthetic polymer: bio-polymer is between 1:0.7 and 1:2.

3. Incorporation of Bioactive Agents into the Bio-Synthetic Matrix

Bioactive agents can be optionally incorporated into the matrix either by covalent attachment (or “grafting”) to the synthetic polymer through the pendant cross-linking groups, or by encapsulation within the matrix.

Non-limiting examples of bioactive agents that may be incorporated into the matrix by cross-linking include, for example, growth factors, retinoids, enzymes, cell adhesion factors, extracellular matrix glycoproteins (such as laminin, fibronectin, tenascin and the like), hormones, osteogenic factors, cytokines, antibodies, antigens, and other biologically active proteins, certain pharmaceutical compounds, as well as peptides, fragments or motifs derived from biologically active proteins.

When the bioactive agent is grafted onto the polymer, it can either be attached through a pendant cross-linking group on the synthetic polymer or it can be cross-linked to the synthetic or bio-polymer by means of cross-linking agents known in the art, such as N-(3-dimethylaminopropyl)-N′-ethylcarbodiimide (EDC) or N-hydroxysuccimide. For covalent attachment of a bioactive agent, the synthetic polymer is first reacted with the bioactive agent and a cross-linking agent of required and then subsequently cross-linked to the bio-polymer as described above. Methods for cross-linking bioactive agents to polymers are known in the art.

In one embodiment of the present invention, the bioactive agent is covalently attached (grafted) to the synthetic polymer through pendant cross-linking groups on the latter. Suitable bioactive agents for grafting to the polymer are those which contain either primary amino or thiol groups, or which can be readily derivatised so as to contain these groups.

Bioactive agents which are not suitable for grafting to the polymer can be entrapped in the final matrix. For entrapment, the bioactive agent is added to a solution of the synthetic polymer in an appropriate solvent prior to mixture of the synthetic polymer and the bio-polymer to form a cross-linked hydrogel. Alternatively, the bioactive agent can be added to a solution containing both the synthetic and bio-polymers prior to the cross-linking step. The bioactive agent is mixed into the polymer solution such that it is substantially uniformly dispersed therein, and the hydrogel is subsequently formed as described above. Appropriate solvents for use with the bioactive agent will be dependent on the properties of the agent and can be readily determined by one skilled in the art.

4. Entrapment of Cells in the Bio-Synthetic Matrix

The bio-synthetic matrix according to the present invention may also comprise cells entrapped therein to permit outgrowth of the cells to form an artificial tissue in vitro. A variety of different cell types may be incorporated into the bio-synthetic matrix, for example, myocytes, adipocytes, fibromyoblasts, ectodermal cells, muscle cells, osteoblasts (i.e. bone cells), chondrocytes (i.e. cartilage cells), endothelial cells, fibroblasts, pancreatic cells, hepatocytes, bile duct cells, bone marrow cells, neural cells, genitourinary cells (including nephritic cells), or combinations thereof. Totipotent stem cells, pluripotent or committed progenitor cells or re-programmed (dedifferentiated) cells can also be encapsulated in the matrix and stimulated to produce a certain cell line by contact with one or more appropriate activating compound(s) as is known in the art.

Cells can be readily entrapped in the final matrix by addition of the cells to a solution of the synthetic polymer prior to admixture with the bio-polymer to form a cross-linked hydrogel. Alternatively, the cells can be added to a solution containing both the synthetic and bio-polymers prior to the cross-linking step. The synthetic polymer may be reacted with a bioactive agent prior to admixture with the cells if desired. Typically, for the encapsulation of cells in the matrix, the various components (cells, synthetic polymer and bio-polymer) are dispersed in an aqueous medium, such as a cell culture medium or a diluted or modified version thereof. The cell suspension is mixed gently into the polymer solution until the cells are substantially uniformly dispersed in the solution, then the hydrogel is formed as described above.

5. Other Elements

The present invention also contemplates the optional inclusion of one or more reinforcing material in the bio-synthetic matrix to improve the mechanical properties of the matrix such as the strength, resilience, flexibility and/or tear resistance. Thus, the matrix may be reinforced with flexible or rigid fibres, fibre mesh, fibre cloth and the like. The use of such reinforcing materials is known in the art, for example, the use of fibres, cloth, or sheets made from oxidised cellulose or polymers such as polylactic acid, polyglycolic acid or polytetrafluoroethylene for medical applications is known.

The reinforcing material can be incorporated into the matrix using standard protocols. For example, an aqueous solution of synthetic and bio-polymers in an appropriate buffer can be added to a fibre cloth or mesh, such as Interceed (Ethicon Inc., New Brunswick, N.J.). The aqueous solution will flow into the interstices of the cloth or mesh prior to undergoing cross-linking and will thus form a hydrogel with the cloth or mesh embedded therein. Appropriate moulds can be used to ensure that the fibres or fibre mesh are contained entirely within the hydrogel if desired. The composite structure can subsequently be washed to remove any side products generated during the cross-linking reaction. Typically, the fibres used are hydrophilic in nature to ensure complete wetting by the aqueous solution of polymers.

6. Preparation of Artificial Tissue

In accordance with the present invention, artificial tissue is constructed by the association of cells with a suitable bio-matrix scaffold. Association of cells with (i.e. growth of cells over and/or into) the bio-synthetic matrix scaffold can be readily achieved in vitro using standard cell culture techniques. For example, cells from one or more appropriate cell lines, such as human endothelial or epithelial cells, can be seeded either directly onto the matrix or onto an appropriate material surrounding the matrix. After growth in the presence of a suitable culture medium for an appropriate length of time, histological examination of the matrix can be conducted to determine whether the cells have grown over the surface of and/or into the matrix. Alternatively, if cells have been encapsulated in the bio-matrix, the matrix can be cultured in an appropriate medium and out-growth of the cells can be assessed after a suitable time. The present invention contemplates a variety of cell lines for this purpose. Typically cell lines with extended lifespans, such as immortalised cell lines, are used. The use of vascular cell lines, such as human vascular endothelial cells, can allow the development of blood vessel-like structures in or on the artificial tissue. One skilled in the art will appreciate that the cell line will be selected depending upon what type of tissue is being emulated

7. Testing of the Bio-synthetic Matrix and/or Artificial Tissue

It will be readily appreciated that the bio-synthetic matrix must be non-cytotoxic in order to be suitable for use as a scaffold for artificial tissue construction. The cytotoxicity of the bio-synthetic matrix can be assessed using standard techniques such as the Ames assay to screen for mutagenic activity, the mouse lymphoma assay to screen for the ability of the matrix to induce gene mutation in a mammalian cell line, in vitro chromosomal aberration assays using, for example, Chinese hamster ovary cells (CHO) to screen for any DNA rearrangements or damage induced by the matrix. Other assays include the sister chromatid assay, which determines any exchange between the arms of a chromosome induced by the matrix and in vitro mouse micronucleus assays to determine any damage to chromosomes or to the mitotic spindle. Protocols for these and other standard assays are known in the art, for example, see OECD Guidelines for the Testing of Chemicals and protocols developed by the ISO.

If desired, physical properties of the bio-synthetic matrix such as the LCST and permeability can be tested. The LCST of the bio-synthetic hydrogel matrix can be measured using standard techniques. For example, LCST can be calculated by heating samples of the matrix at about 0.2° C. per minute and visually observing the cloud point (see, for example, H. Uludag, et al., J. Appl. Polym. Sci. 75:583-592 (2000)). Permeability of the bio-synthetic matrix can be determined by assessing the average pore sizes for the matrix using standard techniques such as glucose and PBS permeability assessment using a permeability cell and/or atomic force microscopy. In accordance with one embodiment of the present invention, the bio-synthetic matrix has an average pore size between about 90 nm and about 500 nm. In another embodiment, the matrix has an average pore size between about 10 nm and about 300 nm.

The ability of the matrix to support cell growth can also be assessed in vitro using standard techniques. For example, cells from an appropriate cell line, such as human epithelial cells, can be seeded either directly onto the matrix or onto an appropriate material surrounding the matrix. After growth in the presence of a suitable culture medium for an appropriate length of time, histological examination of the matrix can be conducted to determine whether the cells have grown over the surface of and/or into the matrix.

A suitable cell line can be selected to determine whether the matrix can support angiogenesis, for example, immortalised human umbilical vein endothelial cells (HUVECs) may be employed for this purpose. The ability of the matrix to support in-growth of HUVECs or proliferation and migration of HUVECs embedded within the matrix resulting in the formation of vessel tubes or cords is indicative of the ability of the matrix to support angiogenesis.

The matrix and/or the culture medium can optionally be supplemented with growth factors to promote in-growth, proliferation and/or migration of cells as is known in the art.

The ability of the matrix to support in-growth of nerve cells can also be assessed in vitro. For example, a nerve source, such as dorsal root ganglia, can be embedded into an appropriate material surrounding the matrix or directly inserted into the matrix. Alternatively, holes can be formed in the hydrogel and subsequently filled with plugs of an appropriate material comprising the nerve source. An example of a suitable material would be a soft collagen based gel. Cells from an appropriate cell line can then be seeded either directly onto the matrix or onto an appropriate material surrounding the matrix and the matrix can be incubated in the presence of a suitable culture medium for a pre-determined length of time. Examination of the matrix, directly and/or in the presence of a nerve-specific marker, for example by direct observation or by immunofluorescence using a nerve-specific fluorescent marker and confocal microscopy, for nerve growth will indicate the ability of the matrix to support neural in-growth.

The nerve cells can be analysed for the presence of sodium channels. Since sodium channels are integral to the generation of action potentials, their presence in nerve cells and fibres provides an indication that the in-grown nerves are functional. The presence of sodium channels in the nerve cells can be determined, for example, by immunohistochemical techniques carried out on the artificial tissue. Sodium channel antibodies are commercially available and can be employed for this purpose either alone or in conjunction with a labelled secondary antibody.

The functionality of the in-grown nerve cells in the bio-synthetic matrix or in artificial tissue can be tested by techniques known in the art. For example, functionality can be measured by the ability of nerves to generate action potentials. Action potentials (AP) propagate from axons to the nervous system to cause pain, and also to the nerve terminals within epithelium to cause the release of neuropeptides. Thus, the functionality of the in-grown nerve cells can be measured by direct electrophysiological recording of action potentials in the nerve cells growing into the bio-synthetic matrix, using standard methods known to a worker skilled in the art. For example, evaluating the recording profile and/or the conduction velocity of the AP can be used to assess function and/or possible nerve toxicity. The functionality of the nerve cells can also be determined by analysing for the release of neuropeptides. For example, the release of the neuropeptide substance P(SP) in response to a suitable stimulus, such as application of a neurotoxin, can be determined by evaluation in a dose and/or time dependent fashion. Methods of stimulating release of neuropeptides and analysing for their presence are known in the art. Kits comprising reagents for this purpose are also commercially available.

Method of Innervation

The artificial tissue according to the present invention is innervated. A method of innervation is provided that can be applied to the artificial tissues comprising the bio-synthetic matrix of the present invention as well as other artificial tissues known in the art.

Innervation of the bio-synthetic matrix based artificial tissue as described above, or other artificial tissues or tissue substitute is achieved by culturing the “tissue” under appropriate conditions in the presence of a nerve source. Examples of suitable nerve sources include, but are not limited to, dorsal root ganglia, trigeminal ganglion, and human or rodent nerve cell lines. These nerve sources can be embedded into the artificial tissue or into an appropriate material surrounding the tissue. The artificial tissue is incubated in the presence of a suitable culture medium for an appropriate length of time to permit neural growth. The culture medium and/or the bio-synthetic matrix may contain additional substances known in the art to promote nerve growth, for example, additional nutrients, growth supplements, growth factors, differentiating factors and the like. In one embodiment of the present invention, nerve growth factor, retinyl acetate, retinoic acid, or a combination thereof are used to promote nerve growth. In another embodiment, laminin can be incorporated within the matrix to promote nerve cell in-growth. For example, a laminin gradient can be created within the matrix to promote the directional growth of nerves. Protease inhibitors may also be used to prevent degradation of the bio-synthetic matrix. Examination of the tissue, directly and/or in the presence of a nerve-specific marker, for example by immunofluorescence using a nerve-specific fluorescent marker and confocal microscopy, will indicate the extent of neural in-growth.

Applications

The innervated artificial tissues according to the present invention can be used as in vitro alternatives to animals in the toxicological and irritancy testing of a variety of products including, but not limited to pharmaceuticals, diagnostics, household products, cosmetics, personal care products and industrial products. The tissues can also be used as models for the therapeutic trials prior to in vivo experimentation. Such models are also important in tailoring pharmaceuticals, for example to minimise degradation into cytotoxic secondary metabolites.

The artificial tissues of the present invention can be used as part of in vitro systems suitable for simulation of pathogenetic and infectious processes, for establishing models of diseases, and for testing active substances.

The artificial tissues can also be used as research tools for investigation of the role of nerves in the various processes, such as wound healing.

The present invention contemplates that the artificial tissue can be tailored for specific applications depending on the type of cell line(s) that is used in conjunction with the bio-synthetic matrix. There are many potential uses of artificial tissue for different mammalian systems. By way of example, small intestine tissue can be used as a model for the molecular and clinical treatment of diseases such as inflammatory bowel disease (Crohn's disease, ulcerative colitus), malabsorptive syndromes (short-gut syndrome), numerous infectious diseases and tumours of the small bowel. In addition, mammalian structural tissue, such as a cartilage model of high fidelity, is important in clinical studies. There are numerous maladies associated with cartilage, including but not limited to knee-joint injuries, back injuries, articular-surface injuries, inflammatory diseases such as arthritis and temporal-mandibular joint disease. Beyond the diseases are the natural processes of maturation through puberty and the geriatric inability to repair and maintain articular surfaces. A suitable tissue model thus would be beneficial for the analysis and development of therapeutic protocols.

In one embodiment of the present invention, the innervated artificial tissue is formed as an artificial cornea. For this application, the tissue is based on a bio-synthetic matrix designed to have a high optical transmission and low light scattering. For example, bio-synthetic matrices comprising a synthetic poly(NiPAAm-co-AAc) co-polymer, a poly(NiPAAm-co-AAc-co-N-acryloxysuccinimide) terpolymer, or a poly(DMAA-co-N-acryloxysuccinimide) co-polymer cross-linked to collagen have high optical transmission, very low light scattering and are capable of remaining clear up to 55° C. The artificial cornea can be prepared by admixture of the synthetic and bio-polymers and injection of the resultant mixture into a suitable mould. If required, the matrix can be cross-linked at room temperature. The incubation temperature can then be raised to about 37° C. to allow for the formation of the final hydrogel. For artificial corneas formed from the terpolymer, extensive washing is then performed to remove N-hydroxysuccinimide produced by the cross-linking reaction and to terminate any unreacted cross-linking groups remaining in the matrix prior to use. This artificial cornea is suitable for use in ocular eye irritancy tests as a substitute for current animal models.

The artificial cornea can be used to determine the cytotoxicity of test substances to the cells of the artificial cornea by contacting it with the test substance and determining its effect on the cornea. The effect on the cornea may be measured by determining the viability of the cells associated with the artificial cornea. Numerous methods of determining cell viability are available to a worker skilled in the art and include, but are not limited to, the MTT assay, the release of the cytosolic enzyme lactate dehydrogenase (LDH), and release of PGE₂.

3. KITS

The present invention also contemplates kits comprising the components required to prepare an innervated artificial tissue. The kits may comprise a suitable bio-synthetic matrix, cell lines, nerve source, or combinations thereof. The kits may comprise a “ready-made” form of the matrix or they may comprise the individual components required to make the matrix (i.e. the synthetic polymer, with or without attached bioactive agents, and the bio-polymer) in appropriate proportions. The kits may further comprise media, appropriate cell culture additives, containers, solvents, or a combination thereof. Individual components of the kit may be packaged in separate containers. The kit may further comprise instructions for use.

To gain a better understanding of the invention described herein, the following examples are set forth. It should be understood that these examples are for illustrative purposes only. Therefore, they should not limit the scope of this invention in any way.

### EXAMPLES

EXAMPLE 1
PREPARATION AND TESTING OF HYDROGELS

Abbreviations

| | |
|---|---|
| RTT: | rat-tail tendon |
| ddH₂O: | distilled, de-ionised water |
| PBS: | phosphate buffered saline |
| D-PBS: | Dulbecco's phosphate buffered saline |
| AIBN: | azobis-isobutyronitrile |
| NiPAAm: | N-isopropylacrylamide |
| pNiPAAm: | poly(N-iso-propylacrylamide) |
| AAc: | acrylic acid |
| DMAA: | N,N-dimethylacrylamide |
| ASI: | N-acryloxysuccinimide |
| poly(NIPAAm-co-AAc): | copolymer of NiPAAm and AAc |
| poly(NiPAAm-co- | terpolymer of N-isopropylacrylamide, |
| AAc-co-ASI): | (NiPAAm), acrylic acid (AAc) and N- |
| | acryloxysuccinimide (ASI) |
| poly(DMAA-co-ASI): | co-polymer of DMAA and ASI |
| GPC: | gel permeation chromatography |
| NMR: | nuclear magnetic resonance |
| YIGSR: | amide-terminated pentapeptide (tyrosine- |
| | isoleucine-glycine-serine0arginine) |
| FMLP | formyl-Met-Leu-Phe |

All gel matrices that employ collagen described in the Sections set out below used sterile collagen I, such as telocollagen (rat-tail tendon, RTT) or atelocollagen (bovine or porcine), which can be prepared in the laboratory or more conveniently is available commercially (for example, from Becton Dickinson at a concentration of 3.0-3.5 mg/ml in 0.02N acetic acid for RTT and in 0.012N hydrochloric acid for bovine and porcine collagens). Such collagens can be stored for many months at 4° C. In addition, such collagen solutions may be carefully concentrated to give optically clear, very viscous solutions of 3-30 wt/vol % collagen, suitable for preparing more robust matrices.

Collagen solutions are adjusted to physiological conditions, i.e. saline ionic strength and pH 7.2-7.4, through the use of aqueous sodium hydroxide in the presence of phosphate buffered saline (PBS). PBS, which is free of amino acids and other nutrients, was used to avoid depletion of cross-linking reactivity by side reactions with —NH₂ containing molecules.

1.1 Preparation of a Collagen-pNiPAAm Hydrogel

PNiPAAm homopolymer powder is available commercially (for example, from Polyscience). All other polymers were synthesized as outlined below. A 1 wt/vol % solution of pNiPAAm homopolymer in ddH₂O was sterilised by autoclaving. This solution was mixed with sterile RTT collagen solution [3.0-3.5 mg/ml (w/v) in acetic acid (0.02N in water] (1:1 vol/vol) in a sterile test tube at 4° C. by syringe pumping to give complete mixing without bubble formation. Cold mixing avoids any premature gelification or fibrilogenesis of the collagen. The collagen-pNiPAAm was then poured over a plastic dish (untreated culture dish) or a mould (e.g. contact lens mould) and left to air-dry under sterile conditions in a laminar flow hood for at least 2-3 days at room temperature. After drying to constant weight (˜7% water residue), the formed matrix was removed from the mould. Removal of the matrix from the mould is facilitated by soaking the mould in sterile PBS at room temperature. Continued soaking of the free sample in this solution gives a gel at physiological pH and ionic strength, suitable for cell growth.

1.2 Preparation of a Collagen-Terpolymer Hydrogel

1.2.1. Preparation of a Synthetic Terpolymer

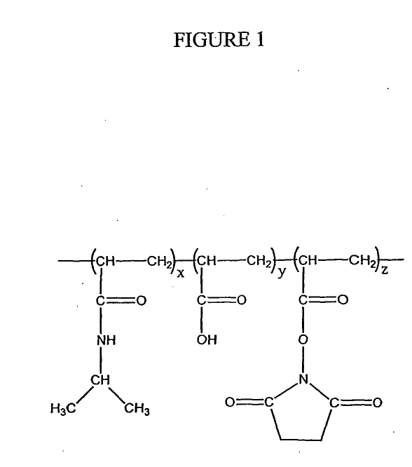

A collagen-reactive terpolymer, poly(NiPAAm-co-AAc-co-AS) (FIG. 1), was synthesised by co-polymerising the three monomers: N-isopropylacrylamide, (NiPAAm, 0.85 mole), acrylic acid (AAc, 0.10 mole) and N-acryloxysuccinimide (ASI, 0.05 mole). The feed molar ratio was 85:10:5 (NiPAAm: AAc: ASI), the free-radical initiator AIBN (0.007 mole/mole of total monomers) and the solvent, dioxane (100 ml), nitrogen purged before adding AIBN. The reaction proceeded for 24 h at 65° C.

After purification by repeated precipitation to remove traces of homopolymer, the composition of the synthesised terpolymer (82% yield) was found to be 84.2:9.8:6.0 (molar ratio) by proton NMR in THF-D_{g}. The Mₙ and M_{w} of the terpolymer were 5.6×10⁴ Da and 9.0×10⁴ Da, respectively, by aqueous GPC.

A solution of 2 mg/ml of the terpolymer in D-PBS remained clear even up to 55° C., consistent with a high LCST. A solution of 10 mg/ml in D-PBS became only slightly cloudy at 43° C. Failure to remove homopolymer formed in the batch polymerisation reaction (due to the NiPAAm reactivity coefficient being greater than that of AAc or ASI) from the terpolymer gave aqueous solutions and hydrogels which cloud at ˜32° C. and above.

1.2.2. Preparation of the Collagen-Terpolymer Hydrogel

A cross-linked, terpolymer-collagen hydrogel was made by mixing neutralised 4% bovine atelocollagen (1.2 ml) with the terpolymer prepared in Section 1.2 [0.34 ml (100 mg/ml in D-PBS)] by syringe mixing at 4° C. (collagen:terpolymer 1.4:1 w/w). After careful syringe pumping to produce a homogeneous, optically clear, bubble-free solution, aliquots were injected into plastic, contact lens moulds and incubated at room temperature (21° C.) for 24 hours to allow reaction of the collagen —NH₂ groups with ASI groups as well as the slower hydrolysis of residual ASI groups to AAc groups. The moulded samples were then incubated at 37° C. for 24 hours in 100% humidity environment, to give a final hydrogel. The hydrogel contained 95.4±0.1% water, 2.3% collagen and 1.6% terpolymer. Matrices were moulded to have a final thickness between either 150-200 μm or 500-600 μm. Each resulting hydrogel matrix was removed from its mould under PBS solution and subsequently immersed in PBS containing 1% chloroform and 0.5% glycine. This wash step removed N-hydroxysuccinimide produced in the cross-linking reaction, terminated any unreacted ASI groups in the matrix, by conversion to acrylic acid groups and sterilised the hydrogel matrix.

Succinimide residues left in the gels prepared from collagen and terpolymer were below the IR detection limit after washing.

1.3 Preparation of a Hydrogel Comprising a Bioactive Agent

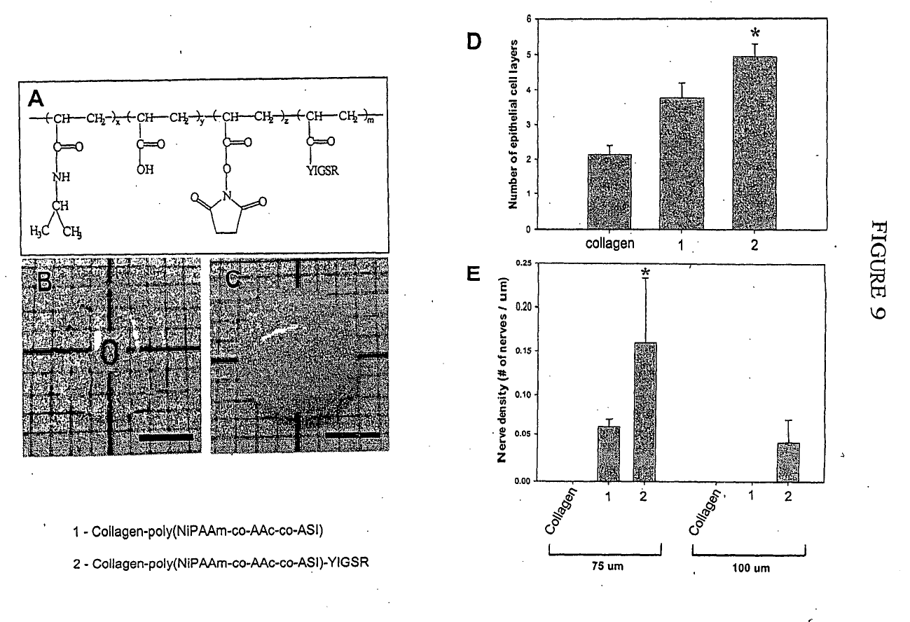

A terpolymer, containing the pentapeptide YIGSR (a nerve cell attachment motif), was synthesised by mixing the terpolymer prepared in Section 1.2 (1.0 g) with 2.8 μg of laminin pentapeptide (YIGSR, from Novabiochem) in N,N-dimethyl formamide. After reaction for 48 h at room temperature (21° C.), the polymer product was precipitated out from diethyl ether and then vacuum dried. ASI groups remaining after reaction with the pentapeptide are available for subsequent reaction with collagen. The structure of this polymer is shown in FIG. 9A.

Cross-linked hydrogels of collagen-terpolymer comprising YIGSR cell adhesion factor were prepared by thoroughly mixing viscous, neutralised 4% bovine collagen (1.2 ml) with terpolymer to which laminin pentapeptide (YIGSR) was covalently attached (0.14 ml, 100 mg/ml) at 4° C., following the procedure described in Section 1.2.2.

The YIGSR content of extensively washed gels was 4.3×10⁻¹¹ mole/ml of hydrated gel (2.6×10⁻⁸ g/ml), quantified by labelling the primary amine-containing tyrosine residue of YIGSR with ¹²⁵I using the Iodogen method and measuring the radioactivity of the incorporated iodine with a standardised gamma counter.

1.4 Comparison of the Physical Properties of Hydrogel Matrices

Collagen thermogels are frail, readily collapse and break, and are obviously opaque (see FIG. 9C). Collagen thermogels were prepared as follows. A sterile RTT collagen solution [3.0-3.5 mg/ml (w/v) in acetic acid (0.02N in water] (1:1 vol/vol) was neutralised with dilute NaOH solution at 4° C., using syringe mixing to homogenise. This neutral solution was injected into a contact lens mould or a parallel plate glass mould. Moulds were then incubated at 21° C. for 24 h, then at 37° C. to spontaneously form translucent thermogels (produced by self association of collagen triple helices into micro-fibrils). The soft matrix was removed from the mould, facilitated by soaking the mould in sterile PBS at room temperature. Continued soaking of the free sample in this solution saturated with chloroform gave an opaque, sterile gel, suitable for cell growth.

The permeability coefficient of glucose in PBS (pH 7.4) through hydrogels prepared as described in Examples 1.3 was calculated from measurements in a permeation cell by periodically removing aliquots of permeate, adding adenosine triphosphate and converting glucose to glucose-6-phosphate with the enzyme hexokinase. The latter was reacted with nicotinamide adenine dinucleotide in the presence of dehydrogenase and the resultant reduced dinucleotide quantified by its UV absorption at 340 nm in solution (Bondar, R. J. & Mead, D. C. (1974) Clin Chem 20, 586-90). Topographies of hydrogel surfaces, fully immersed in PBS solution, were examined by atomic force microscopy (Molecular Image Co., USA) in the “contact” mode. Pore sizes from this technique were compared with average pore diameters calculated from the PBS permeability of the hydrogels as previously described (Bellamkonda, R., Ranieri, J. P. & Aebischer, P. (1995) J Neurosci Res 41, 501-9). The hydrogels had refractive indices (1.343±0.003) comparable to the tear film (1.336-1.357) in the human eye (Patel, S., Marshall, J. & Fitzke, F. W., 3rd (1995) J Refract Surg 11, 100-5). They showed high optical clarity compared to matrices that contain only collagen (FIGS. 9B and C). The hydrogels had pore diameters of 140-190 nm (from both atomic force microscopy and PBS permeability) and a glucose diffusion permeability coefficient of 2.7×10⁻⁶ cm²/s, which is higher than the value for the natural stroma (˜0.7×10⁻⁶ cm²/s, calculated from published diffusion (2.4×10⁻⁶ cm²/s) and solubility (0.3) coefficients (McCarey, B. E. & Schmidt, F. H. (1990) Curr Eye Res 9, 1025-39)).

The following properties of the hydrogels prepared as described in Sections 1.4 and 1.5 indicate that they are cross-linked:
water insoluble,
strong enough to support surgical manipulation with suture thread and needle
relatively flexible in handling
demonstrate an increase in stress at failure and apparent modulus during tensile testing by over x2 on going from —NH₂/ASI equivalent ratio of 0.5 to 1.5.

The hydrogels prepared as described in Section 1.4 and 1.5 have high optical transmission and very low light scattering, comparable to the human cornea, as measured with a custom-built instrument that measures transmission and scatter [Priest and Munger Invest. Ophthalmol. Vis. Sci. 39: S352 (1998)]. In contrast, collagen-pNiPAAm homopolymer gels (as described in Section 1.1; 1.0:0.7 to 1.0:2.0 wt/wt) were opaque at 37° C. In addition, the pNiPAAm homopolymer and collagen in gels from Section 1.1 tend to extract out into aqueous media, including physiological liquids.

1.5 Preparation of a Collagen-Poly(DMAA-co-ASI) Hydrogel

1.5.1. Preparation of a Synthetic Poly(MAA-co-ASI) Co-Polymer

A poly(DMAA-co-ASI) co-polymer was synthesised by co-polymerization of the monomers: N,N-dimethyl acrylamide, (DMAA) and N-acryloxysuccinimide (ASI). The feed molar ratio was 95:5 (DMAA:ASI). The free-radical initiator AIBN and the solvent, dioxane, were nitrogen purged prior to use and polymerisation reaction proceeded at 70° C. for 24 hours.

After purification by repeated precipitation to remove traces of homopolymer, the composition of the synthesized copolymer (70% yield) was found to be 94.8:5.2 (molar ratio) by proton NMR. Molecular mass (Mₙ) was determined at 4.3×10⁴, by aqueous GPC. Polydispersity (PD)=1.70 was also determined by GPC.

A poly(DMAA-co-ASI) co-polymer with the pentapeptide YIGSR covalently attached to unreacted ASI groups was prepared following the protocol outline in Section 13 using the poly(DMAA-co-ASI) co-polymer synthesized as described above.

1.5.2. Preparation of the Hydrogel

A cross-linked collagen-co-polymer hydrogel was prepared by mixing neutralized 5% bovine collagen (1.0 ml) with the synthetic co-polymer prepared in Section 1.5.1. [0.2 ml (200 mg/ml in D-PBS)] by syringe mixing. After careful syringe pumping to produce a homogeneous, bubble-free solution, aliquots were injected into plastic, contact lens moulds and incubated at room temperature for 24 hours to allow reaction of the collagen —NH₂ groups with ASI groups in the co-polymer as well as the slower hydrolysis of residual ASI groups to AAc groups.

The moulded samples were then incubated at 37° C. for 24 hours in a 100% humidity environment to provide the final hydrogel. At gelation, the hydrogel contained 94.8% water, 2.9% collagen and 2.3% synthetic co-polymer. Matrices were moulded to have a final thickness between either 150-200 μm or 500-600 μm. Each resulting hydrogel matrix was removed from its mould under PBS solution and subsequently immersed in PBS containing 1% chloroform and 0.5% glycine. This wash step removed N-hydroxysuccinimide produced in the cross-linking reaction and terminated any residual ASI groups in the matrix, by conversion to acrylic acid groups.

Succinimide residues left in the gels prepared from collagen and copolymer were below the IR detection limit after washing.

Hydrogels comprising collagen and poly(DMAA-co-ASI) co-polymer with the pentapeptide YIGSR covalently bound to the co-polymer were also prepared by this method.

1.6. In Vitro Testing of the Hydrogels: Biocompatibility and Nerve In-growth

A. Immortalized corneal epithelial cells (Araki-Sasaki, K., Aizawa, S., Hiramoto, M., Nakamura, M., Iwase, O., Nakata, K., Sasaki, Y., Mano, T., Handa, H. & Tano, Y. (2000) J Cell Physiol 182, 189-95) were used to evaluate in vitro epithelial coverage on collagen-p(NiPAAm-co-AAc-co-ASI), collagen-p(NiPAAm-co-AAc-co-ASI)-YIGSR and collagen only hydrogels. Hydrogels (500 μm thickness) were embedded on top of a collagen-based matrix that consisted of a mixture of blended neutralized, type I rat-tail tendon collagen (0.3% w/v, Becton-Dickinson, Oakville, Canada) and chondroitin 6-sulfate (1:5 w/w ratio), cross-linked with 0.02% v/v glutaraldehyde (followed by glycine termination of unreacted aldehyde groups) and then thermo-gelled at 37° C. Controls consisted of the collagen matrix alone. Epithelial cells were seeded on top, and constructs were supplemented with a serum-free medium containing epidermal growth factor (Keratinocyte Serum-Free Medium (KSFM; Life Technologies, Burlington, Canada)) until confluence. The medium was then switched to a serum-containing medium (modified SHEM medium (Jumblatt, M. M. & Neufeld, A. H. (1983) Invest Ophthalmol Vis Sci 24, 1139-43)) for 2 days, followed by maintenance at an air/liquid interface. At 2 weeks, constructs were fixed in 4% paraformaldehyde (PFA) in 0.1M PBS and were processed for routine haematoxylin and eosin (H&E) staining.

The number of cell layers and the thickness of the epithelium were measured from 6 random areas for each of 4 samples within each of the 3 experimental groups: control and 2 hydrogels. The epithelium on the collagen-p(NiPAAm-co-AAc-co-ASI)-YIGSR hydrogel was thicker and had a significantly greater (P<0.05) number of cell layers than either collagen-p(NiPAAm-co-AAc-co-ASI) medium or collagen only hydrogels (FIG. 9D).

The hydrogel constructs as described above were also used to examine early nerve in-growth. Dorsal root ganglia (DRG) from chick embryos (E 8.0), were embedded within the surrounding matrix adjacent to the hydrogel. Cultures were supplemented with KSFM medium containing 2% B27 and 1% N2 supplements (Life Technologies) and 1 nM retinyl acetate (Sigma, Oakville, Canada) to support nerve growth. After 4 days, constructs were fixed as described above for immunohistochemistry on whole mounts to visualize nerves within constructs. For nerve immunolocalization, flat mounts were permeabilized with a detergent cocktail (19) (150 mM NaCl, 1 mM ethylenediamine tetraacetic acid, 50 mM Tris, 1% Nonidet P-40, 0.5% sodium deoxycholate, 0.1% sodium dodecyl sulphate), blocked for non-specific staining with 4% foetal calf serum in PBS and incubated in anti-neurofilament 200 antibody (Sigma, Oakville, Canada). They were then incubated with FITC or Cy3-conjugated secondary antibodies (Sigma; Amersham, Baie D'Urfé, Canada, respectively) and visualization by confocal microscopy.

Nerve density (the number of nerves per μm²) was calculated at distances of 75 and 100 μm from the edge of the DRG adjacent to the implant within a 90° pie-shaped wedge extending into the implant. The density (FIG. 9E) of nerves was significantly increased (P<0.05) in the collagen-p(NiPAAm-co-AAc-co-ASI) and collagen-p(NiPAAm-co-AAc-co-ASI)-YIGSR hydrogels compared to collagen only. In addition, the collagen-p(NiPAAm-co-AAc-co-ASI)-YIGSR hydrogels demonstrated an ability to support the growth of nerves that reached 100 μm from the edge of the matrix.

B. Three 12 mm diameter and 650 μm thick discs each of collagen-poly(DMAA-co-ASI)-pentapeptide, collagen-poly(NiPAAm-co-AAc-co-ASI)-pentapeptide hydrogels and a 3% collagen thermogel were soaked for 30 minutes in PBS. They were each laid onto a 12 mm membrane insert commercially available for a culture dish and adhered to the membrane with a thin coating of gelatin. After drying for 10 minutes, 1×10⁴ human corneal epithelial cells (HCEC) in the commercially available keratinocyte serum free medium (KSFM) were added to the top of the gels, and KSFM without cells to the underlying well. Cultures were incubated at 37° C. with 5% CO₂.

Within 12 hours the cells had adhered to the surface of the matrix in all samples. Medium was changed every second day with KSFM added to the inserts, and to the outside wells. HCEC were grown to confluence on the gels and reached confluence on the same day (5 days). The medium in the inserts and surrounding wells was replaced by SHEM. After 2 more days, the medium was removed from the inserts, and the volume of SHEM in the underlying wells reduced to 0.5 ml. The epithelium was allowed to stratify for a further 7 days and the layer of cells visualized.

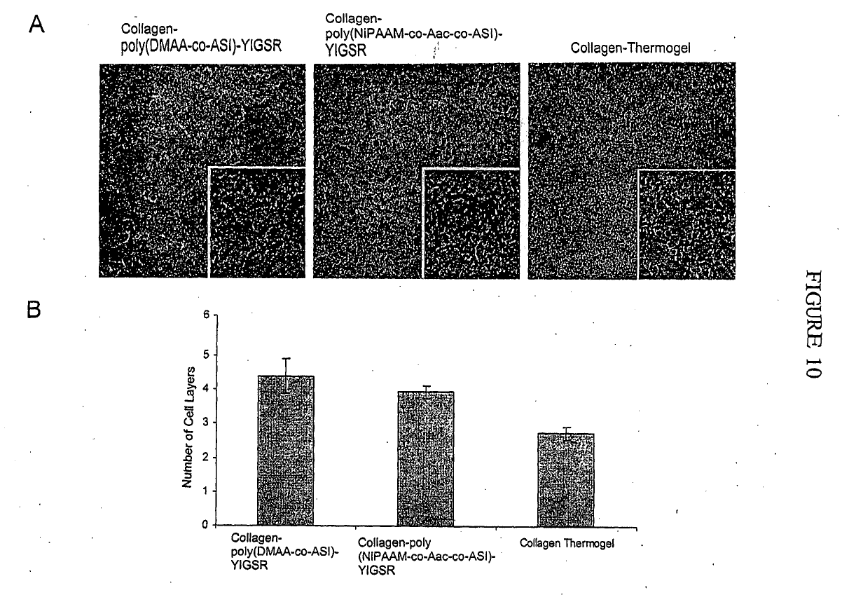

After 7 days, the membranes were fixed in 4% paraformaldehyde in PBS for 30 minutes at 4° C. Samples were prepared for cryosectioning by equilibration in 30% sucrose in PBS followed by flash freezing in a 1:1 mixture of 30% sucrose in PBS and OCT. These were cryosectioned to 10 μm and the structure visualized by Hand E staining. The number of cell layers in the stratified epithelium was determined by counting nuclei and identifying cell borders. While the collagen thermogel attained an epithelial thickness of approximately 2 cells, this is not representative of the human cornea that has an epithelium that contains between 5 and 7 cell layers. HCEC cultured and induced to stratify on poly(DMAA-co-ASI)-YIGSR and poly(NiPAAm-co-AAc-co-ASI)-YIGSR resulted in an epithelium about 4.5 cell layers thick that included apparently keratinized outer layers suggesting appropriate differentiation of the epithelium (FIG. 10).

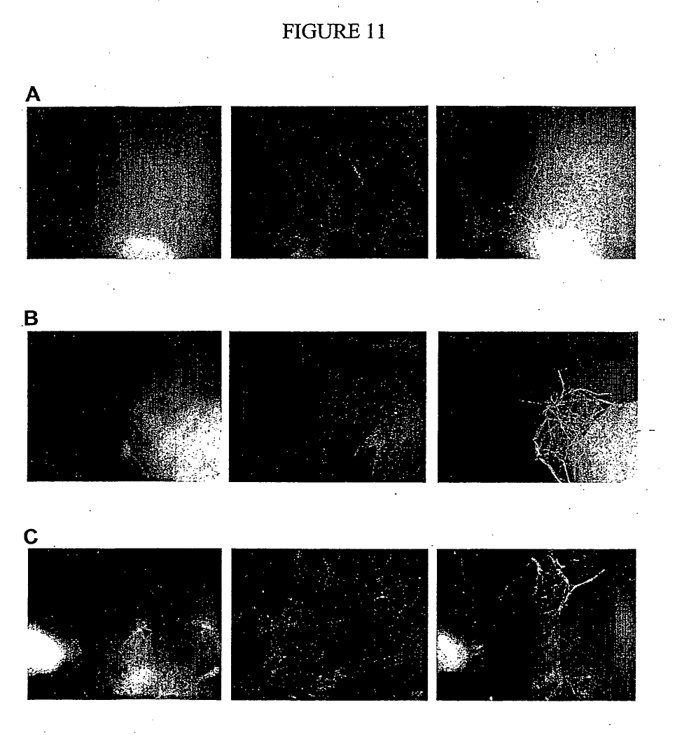

Twelve millimeter diameter and 650 μm thick discs each of collagen-poly(DMAA-co-ASI)-pentapeptide, collagen-poly(NiPAAm-co-AAc-co-ASI)-pentapeptide hydrogels and a 3% collagen thermogel were soaked for 30 minutes in PBS. Discs were laid in a 6 cm culture dish, and four 1 mm holes bored through each. The holes were filled a third of the way up with a plug of 0.3% collagen crosslinked with glutaraldehyde and quenched with glycine. After 10 minutes, dorsal root ganglions from E8 chicks were dipped in the same collagen mixture and placed in the holes. The holes were filled the rest of the way with cross linked collagen, and allowed to set for 30 minutes at 37° C. Cultures were grown for 4 days in KSFM supplemented with B27, N2, and 1 nM retinoic acid for 4 days and neurite extension monitored by brightfield microscopy. The innervated discs were fixed in 4% paraformaldehyde in PBS for 30 minutes room temperature, stained for NF200 immunoreactivity, and visualized by immunofluorescence. Localization was visualized on the surface and in the center of the polymer disc. While there was some neurite extension over the surface of the collagen thermogel, none could be seen extending into the polymer itself. In the collagen-synthetic copolymer with the YIGSR graft, neurites could be seen extending into the polymer matrix. As well, in both the collagen-terpolymer and collagen-synthetic copolymer with YIGSR grafts, extensive innervation could be seen over the surface of the polymers suggesting a better surface innervation than identified with the collagen thermogel (FIG. 11; A depicts the collagen thermogel, B depicts the collagen-p(NiPAAm-co-AAc-co-ASI)-pentapeptide and C depicts the collagen-p(DMAA-co-ASI)-pentapeptide). The left column represents immunofluorescent visualizations of the middle of the polymers stained for the nerve neurofilament marker—NF200. The middle column depicts a brightfield view of the surface of the polymer with the neurites extending from the ganglion source. The right column represents an immunofluorescent visualization of the same surface view of the polymer stained for NF200 immuno-reactivity. The arrows indicate neurites extending in the middle of the polymer. The intact human cornea demonstrates both sub-epithelial surface and deep nerves suggesting that these matrices are both biocompatible to nerves and can emulate the corneal stroma.

### Example 2

### Method for Functional Innervation of an Artificial Tissue

2.1 Tissue Engineering Innervated Corneas and Nerve Growth Patterns

Cell lines with extended lifespans [M. Griffith et al., Science 286, 2169 (1999)] were used to develop tissue engineered (TE) corneas as substrates for nerve innervation. The cell lines included a SV40 immortalized corneal epithelial cell line known to have the appropriate receptors (neurokinin-1, NKI) for the Substance P(SP) neurotransmitter [K. Araki-Sasaki et al., J. Cell Physiol. 182, 189 (2000)] and human papilloma virus (HPV) 16 E6E7 immortalized corneal stroma, corneal endothelial and human umbilical vein endothelial cell lines (HUVECs) [M. Griffith, et al., in Methods in Tissue Engineering, A. Atala, R. P. Lanza, Eds. (Academic Press, San Diego, Calif., 2002), Chap. 9].

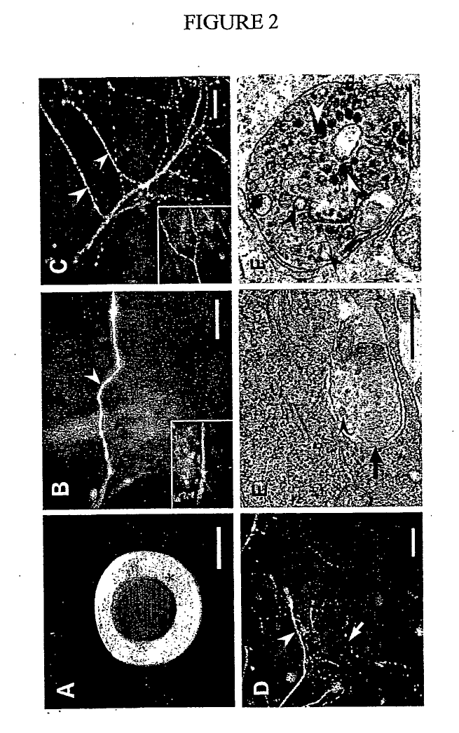

Dorsal root ganglia (DRG) dissected from eight day old chick embryos served as the nerve source. DRG were embedded in an annular, collagen-containing hydrogel that served as a scleral scaffold, within the centre of which a cornea was fabricated (see FIG. 2A). DRG can also be placed within the fabricated cornea.

In more detail, DRG, isolated by collagenase digestion and micro-dissection, were embedded in a ring of neutralised, type I rat tail tendon collagen (0.3% (w/v), Becton-Dickinson) with chondroitin 6-sulfate (1.5% (w/v)) which had been previously cross-linked with 0.02% v/v glutaraldehyde (followed by glycine termination) and thermo-gelled at 37° C. for 2 hours. A cornea was fabricated within this collagen ring, using a blend of neutralised type I rat tail tendon collagen and chondroitin-6-sulphate (Sigma). A laminin (Becton-Dickinson) gradient was created within the stroma to promote the growth of nerves towards the epithelium. Three layers were made with concentrations increasing from bottom to top (0, 10 and 20 μg/ml). This formulation was then cross-linked with 0.02% glutaraldehyde. Residual aldehyde groups were reacted with a 0.8% aqueous glycine (w/v) solution (details in M. Griffith, et al., in Methods in Tissue Engineering, A. Atala, R. P. Lanza, Eds. (Academic Press, San Diego, Calif., 2002), Chap. 9). The construct was then thermo-gelled by incubation at 37° C. for 2 hours. The cultures were supplemented with a modified SHEM medium [M. M. Jumblatt, A. H. Neufeld, Invest. Ophthalmol. Vis. Sci. 24, 1139 (1983)] containing 2% B27 and 1% N2 supplements (Life Technologies). Optimized concentrations of 1 nM retinyl acetate (RA; Sigma) and 100 ng/ml nerve growth factor (NGF; Sigma) were added to the growth medium and upper corneal layer, respectively, to induce nerve in-growth. At epithelial confluence, the constructs were airlifted and maintained at an air-liquid interface for up to 10 days until used.

Whole mount immunofluorescence with nerve specific anti-neurofilament antibody markers was used to analyse the innervation of the artificial cornea Cornea constructs were fixed in 4% paraformaldehyde in 0.1M phosphate buffered saline (PBS), and then permeabilized by treatment with RIPA detergent (150 mM NaCl, 1 mM ethylenediamine tetraacetic acid (EDTA), 50 mM Tris, 1% Nonidet P-40, 0.5% sodium deoxycholate and 0.1% sodium dodecyl sulphate) for 20 minutes. They were rinsed in Tris buffered saline (TBS), and incubated with anti-neurofilament 200 (Sigma; diluted 1:40 in TBS containing 0.6% carrageenan and 0.3% Triton-X 100 (TCT)) over 2 nights at 4° C. The constructs were then rinsed in TBS and incubated with a Cy3-conjugated secondary antibody (1:200 in TCT; Amersham) for 150 minutes at room temperature (RT). Negative controls were incubated without the primary antibody. Positive controls included staining of DRG and neural tube explants. Nerve growth patterns identical to those observed in human corneas were demonstrated in fabricated corneas. Nerves bundles from the DRG within the scleral scaffold coursed through the corneal stroma (FIG. 2B) and bifurcated with successively finer branches to form a plexus (FIG. 2C) below the basal epithelial cells. As in natural corneas, many bundles of this nerve plexus ran parallel to each other with bifurcations running at near right angles. The inset in FIG. 2C shows corresponding deep stromal nerves seen by in vivo confocal microscopy within the human cornea.

Both beaded and smooth nerve fibres from the sub-epithelial network proceeded to target and migrate within the epithelium (FIG. 2D). Transmission electron microscopy (TEM) showed terminal nerve fibres invaginated corneal epithelial cells (FIG. 2E, and at higher magnification in FIG. 2F), as previously described for human corneas [L. J. Müller, L. Pels, G. F. J. M. Vrensen, Invest. Ophthalmol. Vis. Sci. 37, 476 (1996)], suggesting that these cells receive direct innervation.

2.2 Nerve Action Potentials within Tissue Engineered Corneas

Sodium channels are integral to the generation of nerve action potentials. Action potentials propagate from axons to the central nervous system to cause pain, and also to the nerve terminals within the epithelium to cause the release of neuropeptides.

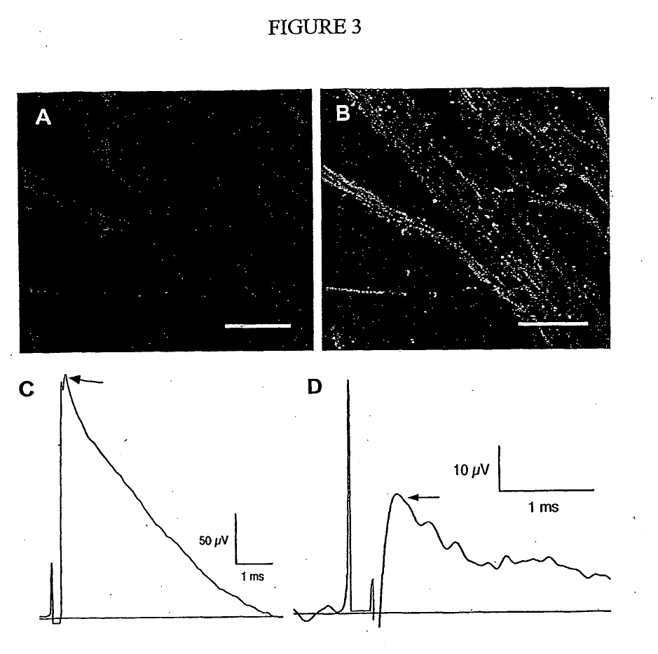

Immunohistochemistry was conducted on the innervated artificial corneas. Briefly, paraformaldehyde-fixed constructs were rinsed in 0.05 M Tris buffer, pH 7.4 and permeabilized in Tris buffer containing 0.3% Triton X-100. Following blocking with 10% normal goat serum in buffer, tissues were incubated overnight at 4° C. with the primary antibody, monoclonal anti-PAN sodium channel antibody (Sigma), at a dilution of 1:250 in Tris buffer containing 2% normal goat serum. The tissues were then rinsed thoroughly in Tris buffer and reacted with a 1:100 dilution of secondary antibody, goat anti-mouse Alexa 488 (Molecular Probes), in Tris buffer for 90 minutes prior to visualization under fluorescence microscopy. Sodium channels were observed in the nerve fibres of the TE corneas (FIG. 3A, B), indicating that the axons most likely possess the machinery necessary to be excitable and functional.

Direct electrophysiological recording from the corneal epithelium was therefore conducted to confirm that nerve bundles growing into the cornea were able to conduct lidocaine-sensitive action potentials that were evoked by stimulation of the ganglion cell cluster (FIG. 3C, D). Tissue engineered corneas were transferred into an interface recording chamber, perfused with artificial saline containing (in mM): NaCl: 126, KCl: 3.0, MgSO₄: 2.0, NaHCO₃: 26, NaH₂PO₄ 1.25, CaCl₂: 2.0, dextrose: 10, oxygenated with 95% O2/5% CO2 at room temperature. Cathodal stimulation of ganglion cell clusters was done using silver wires pressed lightly against the surface and applying square wave stimulus pulses of 50 μs duration and typically 60-80 V in amplitude. Differential recordings of electrical responses from nerve fibre bundles were recorded with glass micropipettes (≈50 μm tips) filled with 150 mM NaCl. Because of the close proximity of the stimulation to the recording electrodes, a very large stimulus artefact was generated that obscured the very small action potentials (FIG. 3C, the action potential is indicted by the arrow).

To observe action potentials in isolation, evoked responses were recorded before and after addition of 50 mM lidocaine HCl. Subtracting the responses in lidocaine from control responses yielded isolated action potentials with the stimulus artefacts largely removed (FIG. 3D, the action potential is indicted by the arrow). The compound action potential shown in FIG. 3D had a short latency and an amplitude of ˜26 μV. The action potentials exhibited a configuration and amplitude similar to those recorded from nerve endings in guinea-pig corneas [J. A. Brock, E. M. McLachlan, C. Belmonte, J. Physiol. 512, 211 (1998)]. The generation of action potentials is important to the function of the corneal nerve endings in the epithelium.

2.3 Effects of Innervation on Wound Healing and Response to Chemicals

The loss of corneal innervation is known to reduce epithelial cell proliferation and to slow wound healing in rabbit corneas. To test whether this effect was reproducible in the artificial cornea system, epithelial wounds were created in TE corneas constructed with and without nerves, and wound closure rates were measured. To create wounds, a circle of filter paper (3 mm diameter) was placed on the epithelium of each construct, allowed to adhere and then peeled off, leaving an area devoid of epithelial cells, as determined by scanning electron microscopy (SEM) on random samples. Wound closure (re-epithelialization) was determined at 0, 6, 12, 18, 24, 36, 48 and 72 hours post-wounding by microscopy, with area calculated using BioRad Quantity One© software. Mean initial wound areas of the innervated group (6.68±0.17 mm²) and non-innervated group (7.02±0.11 mm²) were not significantly different (t-test, P=0.21). To account for variation in original wound sizes, a new healing parameter that is independent of the original wound area was developed. Since wound healing is dependent upon the number of cells at the wound edge that can migrate into the wound or multiply to cover the wound, the number of progenitor cells at the wound edge is proportional to the circumference of the wound. By dividing the change in area of the wound by the original wound circumference, a measure of the healing that has occurred per number of progenitor cells was obtained This new normalised healing parameter is now independent of the original wound area.

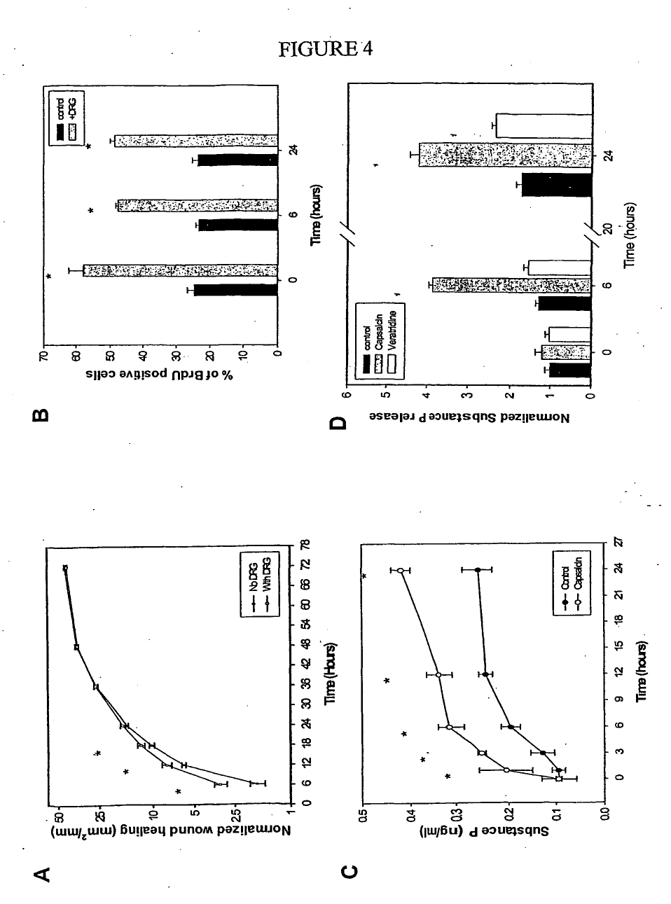

During the first 18 hours, the innervated corneal constructs showed a higher rate of wound closure (see FIG. 4A, which shows normalized total healing (change in wound area (mm²)/original wound circumference (mm)) for TE corneas with and without DRG. At 6, 12 and 18 hours there is significantly faster wound healing for corneas with DRG. Corneas with DRG and controls, n=16. *P<0.05 versus control (2-way ANOVA). By 24 hours, however, no differences in total wound healing were observed between the innervated constructs and non-innervated controls. Bromodeoxyuridine (BrdU, a mitotic indicator) incorporation at 0, 6 and 24 hours post-wounding showed an increase in the percentage of labeled epithelial cells in innervated constructs compared to non-innervated controls (FIG. 4B). The percentage of BrdU-positive cells within each treatment group did not increase over time post-wounding, indicating that epithelial cell proliferation did not increase in the first 24 hours post-injury. All groups, n=3. *P<0.05 versus controls (2-way ANOVA).

These results demonstrate that the presence of nerves in the TE cornea promotes proliferation of epithelial cells, and are consistent with previous in vivo rabbit studies [J. Garcia-Hirschfeld, L. G. Lopez-Briones, C. Belmonte, Exp. Eye Res. 59, 597 (1994)]. However, no significant changes (p>0.05) in the mitotic index were observed within either group over the first 24 hours after wounding. This suggests that the higher rate of wound closure of innervated constructs over the initial 18 hours (FIG. 4A) is most likely due to faster epithelial cell migration. This observation is supported by previous reports that the presence of nerves promotes migration of corneal epithelial cells [R. W. Beuerman, B. Schimmelpfennig, Exp. Neurol 69, 196 (1980)]. These data are also consistent with in vivo rabbit studies that demonstrate increased epithelial cell proliferation begins in the wound area only 24 hours after wounding [L. Gan, H. Hamberg-Nystrom, P. Fagerholm, G. Van Setten, Acta. Ophthalmol. Scand. 79, 488 (2001)].

During cornea wound healing, neuropeptides such as substance P(SP) are released from nerve terminals and are believed to promote healing effects associated with corneal innervation [T. Nishida et al., J. Cell Physiol. 169, 159 (1996); M. Nakamura, et al., Curr. Eye Res. 16, 275 (1997)]. Furthermore, the absence of neuropeptides in corneal nerves has been correlated with delayed corneal wound healing [J. Gallar, et al., Invest. Ophthalmol. Vis. Sci. 31, 1968 (1990)]. SP has been shown to exert a stimulatory effect on corneal epithelial cell proliferation and migration [J. Garcia-Hirschfeld, et al., Exp. Eye Res. 59, 597 (1994); T. Nishida et al., J. Cell Physiol. 169, 159 (1996)] via the NK1 receptor [M. Nakamura et al., Br. J. Pharmacol., 120, 547 (1997)] and to play a role in epithelial cell adhesion.

To elicit a functional response such as SP release, innervated TE cornea constructs were treated with capsaicin or veratridine. Briefly, innervated tissue engineered corneas were treated with a total of 1.5 ml of SHEM containing 8.5% Tween 80 and 1.5% ethanol either alone (control) or with 1) 1% (w/v) capsaicin, or 2) 50 μM veratridine. At 0, 1, 3, 6, 12 and 24 hours post-treatment, culture supernatants were collected, flash frozen in liquid nitrogen and stored at −80° C. Substance P content of the medium was measured using a substance P specific competitive peptide enzyme immunoassay (EIA) kit (Peninsula Laboratories). A significant increase in SP release from nerve axons in capsaicin-treated samples was observed over 24 hours, compared to capsaicin-free controls (FIG. 4C). The release of SP was significantly greater at 1, 3, 6, 12 and 24 hours in capsaicin treated corneas compared to controls. All groups, n 3. *P<0.001 versus control (2-way ANOVA).

Differential SP release was seen when levels of the neuropeptide were compared amongst innervated corneal constructs treated with capsaicin, veratridine or drug vehicle only (FIG. 4D). A significant increase in SP release was observed at 6 and 24 hours post-treatment for capsaicin and after 24 hours for veratridine treatments compared to controls. All groups, n=6. *P<0.05 versus control (3-way ANOVA). At 6 hours post-treatment, only capsaicin elicited a significant increase in SP release, whereas at 24 hours, both capsaicin and veratridine elicited significant increases. Capsaicin is a neurotoxin that depletes SP from peripheral nerve terminal stores by an action potential-independent mechanism that is not fully understood. Veratridine, on the other hand, causes SP release from nerve terminals by opening sodium channels and depolarizing the membrane [J. K Neubert et. al., Brain Res. 871, 181 (2000)]. Both sodium channel-dependent and independent mechanisms of SP release were observed in the innervated cornea model. Nerves growing into the TE cornea were therefore capable of both responding to chemical stimuli and conducting action potentials in a fashion similar to native nerve processes.

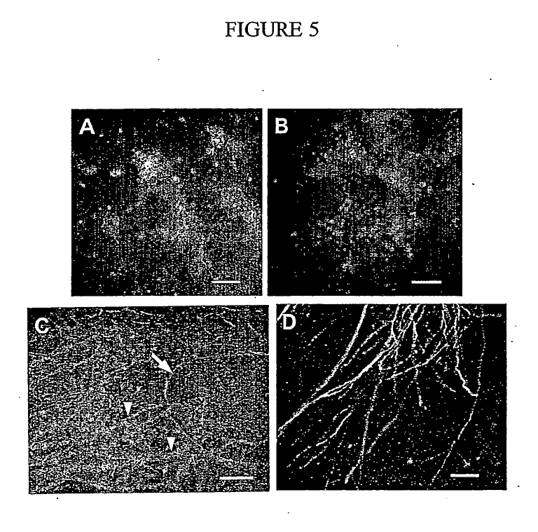

The presence of nerves in the TE cornea was able to protect the epithelium from chemical irritation. Innervated and non-innervated constructs were exposed to a mixture of 8.5% Tween-80 surfactant and 1.5% ethanol in SHEM medium, and live/dead cell counts were performed (FIG. 5A, B, stained with live/dead stain (ethidium bromide and acridine orange). Red indicates dead cells; green indicates live cells). Sixty-two percent of sampled cells were dead in constructs lacking innervation, compared to innervated constructs in which only 11% of cells were dead (n=3 each; p<0.05, t-test). This was consistent with the demonstrated role for nerves in the homeostasis of corneal epithelial cells in the human cornea.

2.4 Collagen-Poly(NiPAAm) Matrix

A collagen-poly(N-isopropyl polyacrylamide) composite was prepared by blending 1% aqueous poly(N-isopropylacrylamide) with 0.3% type I rat tail collagen in 0.02N acetic acid in a 1:1 ratio (v/v). This combined solution was dried down at 20° C. to give a hydrogel, which was then rehydrated in PBS to give approximately 150-200 μm thick hydrogel composites (10% (w/v) total polymers). Use of this matrix in vitro as described above resulted in neurite growth into the polymer scaffold (see FIG. 5D, which shows nerve growth patterns within the matrix as viewed by confocal microscopy. Surface neurites are labelled red, and neurites inside the matrix, labelled green and blue are at depths of 5 μm and 15 μm, respectively.).

### Example 3

### Innervation and Angiogenesis within Artificial Tissues Based on Bio-Synthetic Matrices

3.1 Fibrin-Polyacrylamide Matrix

A fully innervated cornea surrounded by a pseudo-sclera was prepared using a bio-synthetic matrix as described below. To encourage both innervation and angiogenesis, the pseudo-sclera was constructed by adding HUVECs and DRGs into a blended fibrin-polyamide-laminin scaffold. Like the natural cornea and sclera, the cornea was avascular, while the surrounding sclera contained both nerves and blood vessel-like structures (FIG. 5).

3.1.1 Co-polymer Synthesis

The co-polymer, poly(N-isopropylacrylamide-co-acrylic acid) [poly(NiPAAm-co-AAc)], was prepared by conventional free-radical polymerisation of NiPAAm 10.75 g (95 mmol) and acrylic acid 0.36 g (5 mmol) in benzene with azobisisobutyronitrile (AIBN) as the initiator. The reaction can also be conducted in 1,4-dioxane. The product (78% yield) was characterized by GPC (molecular weights: Mₙ=41 O39; M_{w}=70 968; GPC was run in distilled water at 30° C., calibrated with polyethylene glycol standards). ¹H-NMR was used to determine the monomer ratios after the polymer's acrylic groups had been methylated by BF₃-MeOH reagent. This gave a composition of 95.3 mole % NiPAAm and 4.7 mole % AAc after purification by repeated precipitation to remove traces of homopolymer. Very similar values for the purified composition were obtained by back titration. The poly(NiPAAm-co-AAc) at 2 mg/ml has a lower critical solution temperature (LCST) of 54° C. in PBS and 41° C. in ddH₂O. Failure to remove homopolymer formed in the batch polymerization reaction (because of the NiPAAm reactivity coefficient being greater than that of AAc) gives aqueous solutions of the product which cloud at ˜32° C. and above.

A solution of poly(NiPAAm-co-AAc) in ddH₂O can be sterilized by autoclaving or filtering and this solution is stable to storage at room temperature for many months.

3.1.2 Cells and Immortalization

Human umbilical vein endothelial cells (HUVECs) were plated on gelatin-coated tissue-culture dishes in medium 199 supplemented with 10% fetal bovine serum (FBS), 90 mg/l of heparin, 2 mM of L-glutamine and 50 mg/ml endothelial cells growth supplements (ECGS), bFGF (50 ng/ml) and EGF (10 ng/ml) and 10-12 drops of 10 mg/ml of gentamycin (HUVEC medium).

Primary HUVECs were immortalized through viral infection with Human papilloma virus HP16 E6 E7. After 48 hours, the viral supernatants were removed and the medium replaced with the HUVEC medium. After splitting the cells, selection medium (HUVEC medium with 400 μg/ml antibiotic—G418) was added. Cultures were maintained in selective media for 7 days. The G418-selected cells were then grown in HUVEC medium and further expanded.

Human telomerase reverse transcriptase (hTERT) was used to verify the telomerase activity in the immortalized cells. Endothelial cell phenotype was verified by di-acetylated low density lipoprotein (di-Ac-LDL) uptake and binding to an antibody against factor VIII-related antigen as detected by immunocytochemistry.

3.1.3 Fibrin Matrix

Fibrinogen solution (3 mg/ml) was prepared by dissolving fibrinogen in Hank's balanced salt solution (HBSS) with Ca⁺⁺ and Mg⁺⁺. The resultant solution was then sterilized by filtering through a 0.22 μm syringe filter. Thrombin solutions were made by dissolving thrombin in HBSS at a concentration of 1.75 mg/ml.

The fibrinogen solution (3 mg/ml) was mixed with the thrombin solution (1.75 mg/ml) at a ratio of 1:0.03 v/v in wells of different sizes. Within a minute, enzymatic polymerization of fibrinogen gave fibrin gels under gentle agitation at 37° C. To incorporate endothelial cells in the fibrin matrix and to induce angiogenesis, endothelial cells were firstly seeded on the bottom of gelatin-coated wells at high density to provide a confluent monolayer at 48 hours. Then, 5×10⁴ endothelial cells/ml were dispersed in fibrinogen solution prior to polymerization. Fibrin gels were obtained again within a minute. Within 2 weeks of culture at 37° C. with 5% CO₂, tube like vessels were generated within the matrix that associated together in order to form cord structures. These were visible by light microscopy and were counted in order to give an indication of vessel numbers.

3.1.4 Fibrin+P(NiPAAm-Co-AAc)Matrix

Fibrinogen (3 mg/ml) was dissolved in Hank's balanced salt solution (HBSS) with Ca⁺⁺ and Mg⁺⁺ and combined with 0.5% poly(NiPAAm-co-AAc) (NiPAAm:AAC=95:5) in HBSS at a ratio of 1:1, in the presence of thrombin (1.75 mg/ml in HBSS) at a ratio of 1:0.03 v/v to allow polymerization. To incorporate endothelial cells in the matrix and to induce angiogenesis, endothelial cells were first seeded on the bottom of gelatin-coated wells at high density so as to provide a confluent monolayer after 48 hours. Then, 5×10⁴ endothelial cells/ml were dispersed in the solution prior to polymerization as described above.

3.1.5 Identifying Optimal Conditions for Inducing Angiogenesis in the Sclera Model

In producing a scleral model containing blood vessels, a self assembling blood vessel system had to be generated that could be induced to complete itself, that would be limited to the scleral region and not penetrate the central cornea, and could tolerate the medium conditions used both for epithelial stratification and innervation.

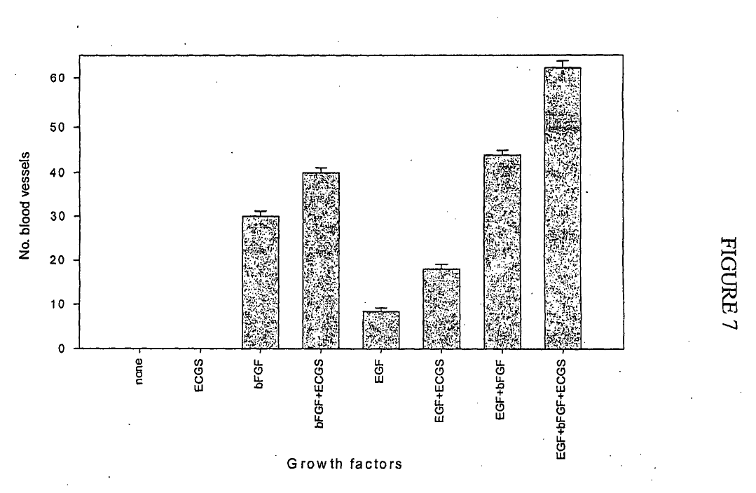

A combinatorial approach was utilized to evaluate what factors are required to achieve the optimal number of blood vessels within this sclera model. The pseudo sclera was generated as per Section 3.1.3. HUVEC blood vessel generation was performed in basic medium 199 using a combinatorial approach that included supplementation with bFGF, ECGS, or EGF. The effectiveness of the growth factors in positively affecting angiogenesis was evaluated by counting the number of tubes identified in a dish by brightfield microscopy. The results suggested that a combination of EGF, bFGF, and ECGS resulted in the greatest number of vessels formed in this system (FIG. 7).

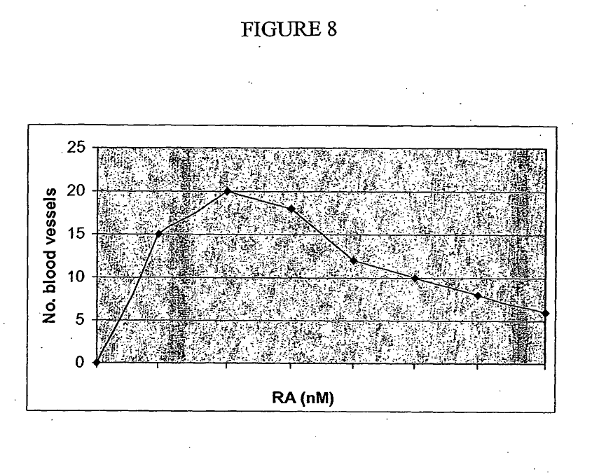

Retinyl acetate is a factor utilized to promote the extension and viability of DRGs in the innervated model. The effect of RA on angiogenesis was evaluated in the system described above. Briefly, blood vessels were induced in the HUVEC model described in Section 3.1.3. The medium of multiple dishes were supplemented with various concentrations of RA, the vessels induced, and cultured as previously described. Blood vessel formation was evaluated by counting the number of vessels within a treated dish. The results are presented in FIG. 8. Blood vessel formation increases in a dose dependent fashion and is optimal at an intermediate range. At high levels the number of blood vessels decreased either due to toxicity or lack of vessel induction. This identified that within the concentrations of RA utilized to induce innervation in the cornea-sclera model, vessel formation may be maintained.

3.1.6 Innervated Cornea and Sclera Model

Dorsal root ganglia were dissected from 8 day old chicken embryos and embedded three-dimensionally inside the Fibrin+poly(NiPAAm-co-AAc) gels with 10 μl/ml of laminin and 10 μl/ml of nerve growth factor (NGF). The construct was supplemented with a modified SHEM medium containing 2% B27, 1 nM retinyl acetate, and 1% N2 supplements (see FIG. 7). The model was generated as described in Sections 2 and 3.1 and was monitored for up to 10 days in culture. The presence of a thickened epithelium, neurite extension, and blood vessel formation was demonstrated. As with the normal human cornea, blood vessel formation and angiogenesis was limited to the sclera and did not penetrate the central cornea. As well, neurites extended both into the central cornea and into the pseudo sclera to target the epithelium. There was no apparent interaction between the newly formed vessels and the neurites as was expected. This suggested that a model that containing all cornea cell types, had optical properties of a normal cornea, and contained nerves and blood vessels could be generated for use in a toxicology model.

3.1.7 Detection of Metalloproteinases

Metalloproteinases MMPs) are a family of closely related zinc-containing enzymes whose principal function is thought to be an integral part of generalized tissue remodeling as well as the formation of new blood vessels. Zymography has the advantage that in addition to detecting enzyme activity it can be used to provide information about the molecular weight of an enzyme and so help identify the enzyme. To achieve this dissolved gelatin is incorporated into a polyacrylamide gel, samples are added to the gel, separated by electrophoresis and the gel allowed to incubate for a while to allow the enzymes to degrade the gelatin. When the gels are stained for proteins clear lysis bands are apparent where the metalloproteinases have degraded the gelatin. The metalloproteases, MMP-2 and MMP-9, were detected in the collagen matrix where neutrophils were added on top of matrix, indicating that FMLP was able to stimulate them (see FIG. 8). This suggests that in a functional sclera model, neutrophils may be supplemented into the sclera with appropriate cues and function appropriately both for creating a path for angiogenesis to occur and to emulate an immune response.

### 3.1.7 CONCLUSIONS

Expression of telomerase activity in immortalized HUVECs was demonstrated. Telomerase activity was not detected in the primary HUVECs indicating that it was possible to immortalize HUVECs to obtain large numbers for use as a cell source for in vitro studies.

Immortalized HUVECs expressed Factor VIII related antigen and took up di-Ac-LDL as markers of endothelial origin. The immortalized HUVECs line ressembled normal HUVECs lines, except that they failed to senesce. The preservation of a normal phenotype in immortalized HUVEC allows use of these cells in tissue engineering to realistically mimic native tissues.

Fibrin, pNiPAAm and poly(NiPAAm-co-AAc) were used to fabricate hydrated matrices for the three-dimensional culture of HUVECs. The hydrogels were able to interact biologically with cells, inducing proliferation and migration (see Table 1). HUVECs are, therefore, able to form blood vessels within the matrices indicating that the polymers have no toxicity towards the cells and support angiogenesis, which is important for cell culture applications. Innervation of the three-dimensional sclera was also demonstrated.

TABLE 1
Blood Vessel Formation in Different Matrices

| Matrix | Presence of Tubes¹ | Longevity² |
|---|---|---|
| Fibrin: 0.5% p(NiPAAm-co-AAc) | | |
| 1:0.1 | ++ | >10 |
| 1:1 | +++ | >10 |
| 1:2 | + | <7 |
| 1:3 | − | <3 |
| Fibrin: 1.0% p(NiPAAm-co-AAc) | | |
| 1:0.1 | ++ | >10 |
| 1:1 | + | >10 |
| 1:2 | + | <7 |
| 1:3 | − | <3 |
| Fibrin: 2.0% p(NiPAAm-co-AAc) | | |
| 1:0.1 | + | <7 |
| 1:1 | + | <7 |
| 1:2 | + | <7 |
| 1:3 | − | <3 |
| Fibrin: 3.0% p(NiPAAm-co-AAc) | | |
| 1:0.1 | + | <7 |
| 1:1 | + | <7 |
| 1:2 | + | <7 |
| 1:3 | − | <3 |
| Fibrin | ++ | >10 |
| Fibrin: 0.5% p(NiPAAm) | | |
| 1:0.1 | | >10 |
| 1:0.2 | ++ | >10 |
| 1:0.3 | + | >10 |

¹+++, ++ and + indicate the presence of blood vessels, with +++ indicating many blood vessels and + indicating few. − indicates that no blood vessels were present.
²as indicated y the occurrence of fibrinolysis

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. An innervated artificial tissue comprising:
(a) a bio-synthetic matrix comprising a synthetic polymer and a biopolymer, said synthetic polymer comprising one or more N-alkyl or N,N-dialkyl substituted acrylamide co-monomer; one or more hydrophilic co-monomer, or one or more acryl- or methaerylcarboxylic acid co-monomer derivatised to contain a pendant crosslinkable moiety, or a combination thereof;
(b) a plurality of non-nerve cells associated with the bio-synthetic matrix; and
(c) a plurality of functional nerve cells associated with the bio-synthetic matrix.

2. The innervated artificial tissue according to claim 1, wherein:
(i) said N-alkyl or N,N-dialkyl substituted acrylaraide co-monomer has a structure of Formula I: 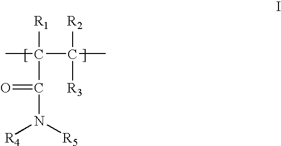
wherein: R₁, R₂, R₃, R₄ and R₅ are independently selected from the group of:
H and lower alkyl;
(ii) said hydrophilic co-monomer has a structure of Formula II: 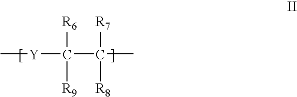
wherein:
Y is 0 or is absent;
R₆, and R₇ are independently selected from the group of: H and lower alkyl;
R₈ is H, lower alkyl or —OR′, where R′ is H or lower alkyl; and
R₉ is H, lower alkyl or —C(O)R₁₀, and
R₁₀ is —NR₄R₅ or —OR″, where R″ is H or CH₂CH₂OH; and
(iii) said acryl- or methacryl-carboxylic acid co-monomer has a structure of Formula III: 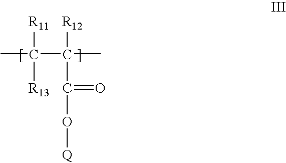
wherein:
R₁₁, R₁₂ and R₁₃ are independently selected from the group of: H and lower alkyl, and
Q is N-succinimido, 3-sulpho-succininido (sodium salt), N-benzotriazolyl, N-imidazolyl and p-nitrophenyl.

3. The innervated artificial tissue according to claim 1, wherein:
(i) said one or more N-alkyl or N,N-dialkyl substituted acrylamide co-monomer is selected from the group of: N-methylacrylamide, N-ethylacrylamide, N-isopropylacrylamide (NiPAAm), N-octylacrylamide, N-cyclohexylacrylamide, N-methyl-N-ethylacrylamide, N-methylmethacrylamide, N-ethylmethacrylamide, N-isopropylmethacrylamide, N,N-dimethylacrylamide, NN-diethylacrylamide, N,N-dimethylmethacrylamide, N,N-diethylmethacrylamide, N,N-dicyclohexylacrylamide, N-methyl-N-cyclohexylacrylamide, N-acryloylpyrrolidine, N-vinyl-2-pyrrollidinone, N-methacryloylpyrrolidine, and combinations thereof;
(ii) said one or more hydrophilic co-monomer is a selected from the group of: acrylic acid, methacrylic acid, 2-hydroxyethyl methacrylate (HEMA), N,N-dimethylacrylamide, N,N-diethylacrylamide, 2-[N,Ndimethylamino]ethylacrylamide, 2-[N,N-diethylamino]ethylacrylamide, N,N-diethylmethacrylamide, 2-[N,N-dimethylamino]ethylmethacrylamide, 2-[N,N-diethylamino]ethylmethacrylamide, N-vinyl-2-pyrrollidinone, 2-[N,N-diethylamino]ethylacrylate, 2-[N,N-dimethylamino]ethylacrylate, 2-[N,N-diethylamino]ethylmethacrylate, 2-[N,N-dimethylamino]ethylmethacrylate, and combinations thereof; and
(iii) said one or more acryl- or methacryl-carboxylic acid co-monomer is selected from the group of: acrylic acid, methacrylic acid, or substituted versions thereof, and said cross-linkable moiety is a succinimidyl group, an imidazole, a benzotriazole, a p-nitrophenol or 2-(N-morpholino)ethanesulphonic acid.

4. The innervated artificial tissue according to claim 3, wherein said synthetic polymer comprises N,N-dimethylacrylamide and N-acryloxysuccinimide.

5. The innervated artificial tissue according to claim 3, wherein said synthetic polymer comprises N-isopropylacrylamide, acrylic acid and N-acryloxysuccinimide.

6. The innervated artificial tissue according to claim 3, wherein said synthetic polymer comprises N-isopropylacrylamide and acrylic acid.

7. The innervated artificial tissue according to claim 1, wherein the biopolymer is selected from the group of: collagens, denatured collagens, gelatin, fibrin-fibrinogen, elastin, glycoprotein, alginate, chitosan, hyaluronic acid, chondroitin sulphate, glycosaminoglycan (proteoglycan), and derivatives thereof.

8. The innervated artificial tissue according to claim 1, wherein said plurality of non-nerve cells are capable of growing as one or more confluent layers over a surface of said bio-synthetic matrix, within the matrix, into the matrix, or a combination thereof.

9. The innervated artificial tissue according to claim 1, wherein said matrix further comprises one or more bioactive agents.

10. The innervated artificial tissue according to claim 9, wherein the bioactive agent is a pentapeptide having the amino acid sequence YIGSR.

11. The innervated artificial tissue according to claim 1, wherein the artificial tissue is formed as an artificial cornea.

12. Use of the innervated artificial tissue according to claim 1 for in vitro toxicity, irritancy or pharmacological testing.

13. Use of a bio-synthetic matrix for the preparation of an innervated artificial tissue, said bio-synthetic matrix comprising a synthetic polymer and a biopolymer, and said synthetic polymer comprising one or more N-alkyl or N,N-dialkyl substituted acrylamide co-monomer; one or more hydrophilic co-monomer, or one or more acryl- or methacryl-carboxylic acid co-monomer derivatised to contain a pendant cross-linkable moiety, or a combination thereof.

14. A method of testing cellular effects of a substance in vitro comprising:
(a) contacting an innervated artificial tissue with a test substance, said artificial tissue comprising
(i) a bio-synthetic matrix comprising a synthetic polymer and a biopolymer;
(ii) a plurality of non-nerve cells associated with the bio-synthetic matrix; and
(iii) a plurality of functional nerve cells associated with the biosynthetic matrix, and
(b) determining the effect of the test substance on said plurality of non-nerve cells, said plurality of functional nerve cells, or both.

15. An in vitro method of toxicology or irritancy testing of a substance comprising:
(a) contacting an innervated artificial tissue with a test substance, said artificial tissue comprising
(i) a bio-synthetic matrix comprising a synthetic polymer and a biopolymer;
(ii) a plurality of non-nerve cells associated with the bio-synthetic matrix; and
(iii) a plurality of functional nerve cells associated with the biosynthetic matrix, and
(b) determining the viability of said plurality of non-nerve cells, said plurality of functional nerve cells, or both.

16. An in vitro method for investigation of the role of nerves in wound healing comprising:
(a) creating a wound in an innervated artificial tissue, said artificial tissue comprising
(i) a bio-synthetic matrix comprising a synthetic polymer and a biopolymer;
(ii) a plurality of non-nerve cells associated with the bio-synthetic matrix; and
(iii) a plurality of functional nerve cells associated with the biosynthetic matrix, said nerve cells being derived from said source, and
(b) comparing wound closure rates in said artificial tissue with wound closure rates in an artificial tissue that is not innervated, or in mammalian tissue.

17. The method according to claim 14, wherein said synthetic polymer comprising one or more N-alkyl or N,N-dialkyl substituted acrylamide co-monomer; one or more hydrophilic co-monomer, or one or more acryl- or methacryl-carboxylic acid co-monomer derivatised to contain a pendant cross-linkable moiety, or a combination thereof.

18. The method according to claim 17, wherein said synthetic polymer comprises N,N-dimethylacrylamide and N-acryloxysuccinimide.

19. The method according to claim 17, wherein said synthetic polymer comprises N-isopropylacrylamide, acrylic acid and N-acryloxysuccinimide.

20. The method according to claim 17, wherein said synthetic polymer comprises N-isopropylacrylamide and acrylic acid.

21. A method for the innervation of an artificial tissue comprising:
(a) providing a source of nerve cells; and
(b) culturing an artificial tissue in a medium in the presence of said source of nerve cells and one or more compounds that promote nerve growth, whereby nerve cells grow from said source into said artificial tissue, wherein said one or more compounds are present in said artificial tissue or in said medium, or both.

22. The method according to claim 21, wherein said one or more compounds that promote nerve growth comprise laminin, nerve growth factor, retinoic acid or retinyl acetate, or a combination thereof.

23. The method according to claim 21, further comprising embedding the source of nerve cells in a matrix prior to step (b).

24. The method according to claim 21, further comprising embedding the source of nerve cells in said artificial tissue prior to step (b).

25. A kit comprising the innervated artificial tissue according to of claim 1 and optionally instructions for use.

26. A kit for the preparation of an innervated artificial tissue comprising:
(a) a bio-synthetic matrix comprising a synthetic polymer and a biopolymer, said synthetic polymer comprising one or more N-alkyl or N,N-dialkyl substituted acrylamide co-monomer; one or more hydrophilic co-monomer, or one or more acryl- or methacrylcarboxylic acid co-monomer derivatised to contain a pendant cross-linkable moiety, or a combination thereof; and
(b) optionally one or more cell lines, a source of nerve cells, instructions for use, or a combination thereof.
